(19) United States
(12) Patent Application Publication
(10) Pub. No.: US 2024/0076709 A1
(43) Pub. Date: Mar. 7, 2024

(54) LEAN PERFUSION CELL CULTURE METHODS

(71) Applicant: AMGEN INC., Thousand Oaks, CA (US)

(72) Inventors: Prince Bhebe, Moorpark, CA (US); Nitya M. Jacob, Oak Park, CA (US)

(21) Appl. No.: 18/241,564

(22) Filed: Sep. 1, 2023

Related U.S. Application Data
(60) Provisional application No. 63/403,896, filed on Sep. 6, 2022.

(51) Int. Cl. C12P 21/00; C07K 16/00

(52) U.S. Cl. CPC C12P 21/005; C07K 16/00; C07K 2317/14

(57) ABSTRACT

Described herein is a low perfusion rate cell culture method for producing recombinant proteins.

### CROSS-REFERENCE TO RELATED APPLICATIONS

This application claims the benefit of priority to U.S. Provisional Patent Application No. 63/403,896, filed Sep. 6, 2022, which is hereby incorporated by reference in its entirety for all purposes.

### FIELD

The present disclosure generally relates to a perfusion culture method for producing recombinant proteins.

### BACKGROUND

Manufacturing of biopharmaceuticals typically begins with culturing mammalian cells engineered to express a desired therapeutic protein. Greater than 50% of approved biologics are produced using mammalian cells, and the number rises every year. Monoclonal antibody biotherapeutics currently represent the largest sector of the biopharmaceuticals market, but new protein modalities having multi-target affinity to treat more diverse therapeutic indications are being tested and approved at an increasing rate. Meeting the growing and complex needs of patients is a driving force for the biopharmaceutical industry. The increasingly complex mix of product modalities in clinical development have made it imperative that manufacturing processes and facilities become even more adaptable, flexible, productive, and cost efficient, while continuing to produce the highest quality biological therapeutics.

For upstream manufacturing processes, process intensification through increasing volumetric productivity is critical to meeting this need. Industrial-scale mammalian cell culture processes have typically included batch, fed batch, and perfusion processes. While these cell culture processes have demonstrated robustness over decades of development and optimization, the ability to intensify is often constrained by process scale limitations such as bioreactor size for batch and fed batch processes and media volume for batching and removal in perfusion processes.

Accordingly, there is a need in the art for novel cell culture methods that facilitate process intensification.

### SUMMARY

Described herein is a novel cell culture method that overcomes certain constraints associated with current, conventional culture methods, enabling process intensification. The method makes efficient use of the bioreactor working volume during the growth and production phases. A low-rate perfusion maintains the culture at a high cell density in at least half the normal bioreactor working volume during the growth phase; the culture volume is then increased to at or near full bioreactor working volume with reduced cell density during the production phase. This transition enables a more efficient culture, reducing feed and waste volumes and increasing titers and productivity.

The present disclosure provides a method for culturing cells to produce a recombinant protein, the method comprising initiating a culture in a bioreactor at a culture volume that is at least 50% of the final bioreactor working volume, inoculating the culture with cells engineered to express the recombinant protein, perfusing the culture at one or more perfusion rates of less than or equal to 0.5 culture volumes/day (V/d) until the culture reaches one or more desired target criteria, increasing the culture volume to a final culture volume, and once the final culture volume is achieved, perfusing the culture at one or more perfusion rates of less than or equal to 0.5 culture volumes/day (V/d) until the culture is terminated or harvested.

In one embodiment, the culture is initiated at a culture volume that is at least 50% to 75% of the final bioreactor working volume. In a related embodiment, the culture is initiated at a culture volume that is at least 60% to 70% of the final bioreactor working volume.

In one embodiment, the culture is initiated at a culture volume that is about 50% to about 75% of the final bioreactor working volume. In a related embodiment, the culture is initiated at a culture volume that is about 60% to about 70% of the final bioreactor working volume.

In another embodiment, the culture is inoculated at a cell density of at least 5×10⁶ cells/mL to about 50×10⁶ cells/mL. In a related embodiment, the culture is inoculated at a cell density of at least 6×10⁶ cells/mL to about 20×10⁶ cells/mL.

In another embodiment, the culture is inoculated at a cell density of about 5×10⁶ cells/mL to about 50×10⁶ cells/mL. In a related embodiment, the culture is inoculated at a cell density of about 6×10⁶ cells/mL to about 20×10⁶ cells/mL.

In another embodiment, the bioreactor is operated in batch mode for up to 24 hours following inoculation. In a related embodiment, the bioreactor is operated in batch mode for about 24 hours following inoculation.

In yet another embodiment, the culture is in a growth phase prior to the increase in culture volume. In a related embodiment, the duration of the growth phase is less than or equal to 60% of the culture duration. In a related embodiment, the culture temperature during the growth phase is 35° C. to 37° C.

In another embodiment, the culture is in a production phase following the increase in culture volume. In a related embodiment, the culture temperature during the production phase is 28° C. to 35° C.

In yet another embodiment, the perfusion rate is at least 0.05 V/d. In another embodiment, the perfusion rate is at least about 0.10 V/d to at least about 0.25 V/d. In a related embodiment, the perfusion rate is at least about 0.15 V/d. In a related embodiment, the perfusion rate is at least about 0.20 V/d. In a related embodiment, the perfusion rate is at least about 0.25 V/d.

In another embodiment, the perfusion rate is about 0.10 V/d to about 0.25 V/d. In a related embodiment, the perfusion rate is about 0.15 V/d. In a related embodiment, the perfusion rate is about 0.20 V/d. In a related embodiment, the perfusion rate is about 0.25 V/d.

In one embodiment, the culture is perfused at one or more perfusion rates of 0.05 V/d to 0.5 V/d (e.g., 0.1 V/d to 0.5 V/d; 0.15 V/d to 0.5 V/d; 0.2 V/d to 0.5 V/d; 0.25 V/d to 0.5 V/d) until the culture reaches one or more desired target criteria, wherein the feed rate(s) and the contemporaneous permeate rate(s) are the same. In a related embodiment, the culture is perfused at a perfusion rate of 0.05 V/d to 0.5 V/d until the culture reaches one or more desired target criteria, wherein the feed rate and the permeate rate are the same.

In one embodiment, once the final culture volume is achieved, the culture is perfused at one or more perfusion rates of 0.05 V/d to 0.5 V/d (e.g., 0.1 V/d to 0.5 V/d; 0.15 V/d to 0.5 V/d; 0.2 V/d to 0.5 V/d; 0.25 V/d to 0.5 V/d) until the culture is terminated or harvested, wherein the feed rate(s) and the contemporaneous permeate rate(s) are the same. In a related embodiment, once the final culture volume is achieved, the culture is perfused at a perfusion rate of 0.05 V/d to 0.5 V/d until the culture is terminated or harvested, wherein the feed rate and the permeate rate are the same.

In another embodiment, a desired target criteria is selected from culture volume, final culture volume, bioreactor working volume, final bioreactor working volume, time point, titer, cell density, packed cell volume, product attribute, process, production and/or a plant schedule. In a related embodiment, the desired target criteria is cell density and/or time post-inoculation. In a related embodiment, the desired target criteria is cell density. In a related embodiment, the desired target criteria is time post-inoculation.

In a related embodiment, the desired target criterion is a cell density of at least 100×10⁵ cells/mL. In another related embodiment, the desired target criterion is a cell density of up to 350×10⁵ cells/mL.

In a related embodiment, the desired target criterion is at least about 24 hours post-inoculation. In yet another related embodiment, the desired target criterion is at least about 48 to 72 hours post-inoculation. In yet another related embodiment, the desired target criterion is about 48 hours to about 72 hours post-inoculation.

In another embodiment, the culture volume is increased using differential perfusion. In a related embodiment, during the differential perfusion, the feed rate is greater than the permeate rate. In another related embodiment, the differential perfusion comprises one or more feed rates and one or more permeate rates. In a related embodiment, at least one feed rate is less than or equal to 0.50 V/d. In yet another related embodiment, at least one feed rate is less than or equal to 0.25 V/d. In another related embodiment, at least one feed rate is less than or equal to 0.50 V/d and the permeate rate is less than or equal to 0.20 V/d. In another embodiment, at least one feed rate is less than or equal to 0.40 V/d and the permeate rate is less than or equal to 0.15 V/d. In yet another related embodiment, the higher feed rate is maintained for at least 6 hours. In another related embodiment, the feed rate is selected to increase the media volume in the bioreactor to a volume of less than or equal to 100% of the bioreactor working volume in the time needed for at least one population doubling of the culture. In another embodiment, the differential perfusion continues until the culture volume in the bioreactor is about 70% to less than or equal to 100% of the final bioreactor working volume.

In another embodiment, the final working volume of the bioreactor is at least 70% to 100% of the bioreactor volume. In a related embodiment, the final working volume of the bioreactor is at least 90% of the bioreactor volume. In a related embodiment, the final working volume of the bioreactor is less than or equal to 100% of the bioreactor volume. In a related embodiment, the final working volume of the bioreactor is about 70%, about 75%, about 80%, about 85%, about 90%, about 91%, about 92%, about 93%, about 94%, about 95%, about 96%, about 97%, about 98%, about 99%, or about 99.5% of the bioreactor volume.

In one embodiment, the cell density at the start of the production phase is at least two (2) times the inoculation cell density. In another related embodiment, the cell density at the start of the production phase is at least 200×10⁵ cells/mL.

In another embodiment, the production phase is at least 10 days. In yet another related embodiment, the production phase is at least 10 to 20 days.

In one embodiment, the bioreactor is connected to a separation system. In a related embodiment, the separation system includes a pumping mechanism and a filter or membrane module. In a related embodiment, the separation system is a tangential flow filtration system. In a related embodiment, the tangential flow filtration system is a recirculating tangential flow filter system, or an alternating tangential flow filtration system.

In one embodiment, the bioreactor volume is 200 liters to 20,000 liters.

In one embodiment, the bioreactor is a single use bioreactor or a stainless-steel bioreactor.

In one embodiment, the cells are mammalian cells, preferably CHO cells.

In one embodiment, the recombinant protein is an antibody, preferably an IgG antibody. In another embodiment, the recombinant protein is an antibody fragment.

The present disclosure also provides a method for culturing cells to produce a recombinant protein, the method comprising initiating a culture in a bioreactor having a culture volume that is at least 50% of the final bioreactor working volume, inoculating the culture with cells engineered to express the recombinant protein at a cell density of at least 5×10⁶ cells/mL, perfusing the culture at one or more perfusion rates of less than or equal to 0.5 culture volumes/day (V/d) until the culture reaches a desired target criteria, increasing the volume of the culture medium to about 70% to less than or equal to 100% of the final bioreactor working volume by differential perfusion wherein the feed rate is greater than the permeate rate; and perfusing the culture at one or more perfusion rates of less than or equal to 0.5 culture volumes/day (V/d) until the culture is terminated or harvested.

The present disclosure also provides a method for producing an isolated, purified, recombinant protein, the method comprising initiating a culture in a bioreactor with a culture volume that is at least 50% of the final bioreactor working volume, inoculating the culture with cells expressing the recombinant protein at a cell density of at least 6×10⁶ cells/mL, maintaining the culture in a growth phase by perfusing the culture at a rate of at least 0.05 V/d until the culture reaches a desired target criteria, increasing the culture volume by differential perfusion until it is at least 70% of the final bioreactor working volume, and maintaining the culture in a production phase by perfusing the culture at a rate of at least 0.05 V/d, harvesting the recombinant protein, processing the recombinant protein through one or more unit operations, and obtaining an isolated, purified, recombinant protein.

In one embodiment, at least one of the one or more unit operations is an affinity chromatography unit operation. In a related embodiment, the affinity chromatography medium is selected from Protein A and immobilized metal affinity chromatography (IMAC) medium.

In one embodiment, at least one of the one or more unit operations is a polishing chromatography unit operation. In a related embodiment, the polishing chromatography medium is selected from anion exchange chromatography media, cation exchange chromatography media, multi-modal chromatography media, hydrophobic interaction chromatography media, and hydroxyapatite chromatography media. In a related embodiment, at least one polishing chromatography medium is operated in bind-and-elute mode, flow-through mode, or frontal mode.

In one embodiment, at least one of the one or more unit operations is selected from virus inactivation, virus filtration, depth filtration, and ultrafiltration/diafiltration (UF/DF).

In one embodiment, the cells are mammalian cells, preferably CHO cells.

In one embodiment, the recombinant protein is an antibody, preferably an IgG antibody. In another embodiment, the recombinant protein is an antibody fragment.

The present disclosure also provides a method for producing a recombinant protein, the method comprising initiating a culture in a bioreactor with cells engineered to express the recombinant protein, culturing the cells by perfusing the culture at a rate of less than or equal to 0.5 V/d to maintain a culture volume of at least 60% of the final bioreactor working volume, subjecting the culture to a feed up phase to increase the culture volume to less than or equal to 100% of the final bioreactor working volume at least 24 hours post-inoculation, following the feed up phase by perfusing the culture at a rate of less than or equal to 0.5 V/d to maintain the culture volume at greater than 70% of the final bioreactor working volume; and harvesting the recombinant protein.

In one embodiment, the cells are mammalian cells, preferably CHO cells.

In one embodiment, the recombinant protein is an antibody, preferably an IgG antibody. In another embodiment, the recombinant protein is an antibody fragment.

Non-limiting example embodiments of the present disclosure also include:
E1. A method for culturing cells to produce a recombinant protein comprising
   initiating a culture in a bioreactor at a culture volume that is at least 50% of the final bioreactor working volume;
   inoculating the culture with cells engineered to express the protein;
   perfusing the culture at one or more perfusion rates of less than or equal to 0.5 culture volumes/day (V/d) until the culture reaches one or more desired target criteria;
   increasing the culture volume to a final culture volume; and
   once the final culture volume is achieved, perfusing the culture at one or more perfusion rates of less than or equal to 0.5 culture volumes/day (V/d) until the culture is terminated or harvested.
E2. The method according to E1, wherein the culture is initiated at a culture volume that is at least 50% to 75% of the final bioreactor working volume.
E3. The method according to E2, wherein the culture is initiated at a culture volume that is at least 60% to 70% of the final bioreactor working volume.
E4. The method according to E1, wherein the culture is inoculated at a cell density of at least 5×10⁶ cells/mL to about 50×10⁶ cells/mL.
E5. The method according to E4, wherein the culture is inoculated at a cell density of at least 6×10⁶ cells/mL to about 20×10⁶ cells/mL.
E6. The method according to E1, wherein the bioreactor is operated in batch mode for up to 24 hours following inoculation.
E7. The method according to E1, wherein the culture is in a growth phase prior to the increase in culture volume.
E8. The method according to E7, wherein the duration of the growth phase is less than or equal to 60% of the culture duration.
E9. The method according to E7, wherein the culture temperature during the growth phase is 35° C. to 37° C.
E10. The method according to E1, wherein the culture is in a production phase following the increase in culture volume.
E11. The method according to E10, wherein the culture temperature during the production phase is 28° C. to 35° C.
E12. The method according to E1, wherein the perfusion rate is at least 0.05 V/d.
E13. The method according to E1, wherein the perfusion rate is at least about 0.10 V/d to at least about 0.25 V/d.
E14. The method according to E1, wherein a desired target criteria is selected from culture volume, final culture volume, bioreactor working volume, final bioreactor working volume, time point, titer, cell density, packed cell volume, product attribute, process, production and/or a plant schedule.
E15. The method according to E14, wherein the desired target criteria is cell density and/or time post-inoculation.
E16. The method according to E14, wherein the desired target criterion is a cell density of at least 100E5 cells/mL.
E17. The method according to E16, wherein the desired target criterion is a cell density of up to 350E5 cells/mL.
E18. The method according to E14, wherein the desired target criterion is at least about 24 hours post-inoculation.
E19. The method according to E18, wherein the desired target criterion is at least about 48 to 72 hours post-inoculation.
E20. The method according to E1, wherein the culture volume is increased using differential perfusion.
E21. The method according to E20, wherein during the differential perfusion, the feed rate is greater than the permeate rate.
E22. The method according to E21, wherein the differential perfusion comprises one or more feed rates and one or more permeate rates.
E23. The method according to E22, wherein at least one feed rate is less than or equal to 0.50 V/d.
E24. The method according to E22, wherein at least one feed rate is less than or equal to 0.25 V/d.
E25. The method according to E22, wherein at least one feed rate is less than or equal to 0.50V/d and the permeate rate is less than or equal to 0.20 V/d.
E26. The method according to E22, wherein at least one feed rate is less than or equal to 0.40V/d and the permeate rate is less than or equal to 0.15V/d.
E27. The method according to E21, wherein the higher feed rate is maintained for at least 6 hours.
E28. The method according to E21, wherein the feed rate is selected to increase the media volume in the bioreactor to a volume of less than or equal to 100% of the bioreactor working volume in the time needed for at least one population doubling of the culture.
E29. The method according to E1, wherein the differential perfusion continues until the culture volume in the bioreactor is about 70% to less than or equal to 100% of the final bioreactor working volume.
E30. The method according to E1, wherein the final working volume of the bioreactor is at least 70% to 100% of the bioreactor volume.
E31. The method according to E10, wherein the cell density at the start of the production phase is at least 2 times the inoculation cell density.
E32. The method according to E10, wherein the cell density at the start of the production phase is at least 200×10⁵ cells/mL.
E33a. The method according to E10, wherein the production phase is at least 10 days.
E33b. The method according to E32, wherein the production phase is at least 10 to 20 days.
E34. The method according to E1, wherein the bioreactor is connected to a separation system.
E35. The method according to E34, wherein the separation system includes a pumping mechanism and a filter or membrane module.
E36. The method according to E34, wherein the separation system is a tangential flow filtration system.
E37. The method according to E36, wherein the tangential flow filter system is a recirculating tangential flow filter system, or an alternating tangential flow filtration system.
E38. The method according to E1, wherein the bioreactor volume is 200 liters to 20,000 liters.
E39. The method according to E1, wherein the bioreactor is single use or stainless-steel.
E40. A method for culturing cells to produce a recombinant protein comprising
   initiating a culture in a bioreactor having a culture volume that is at least 50% of the final bioreactor working volume;
   inoculating the culture with cells engineered to express the protein at a cell density of at least 6×10⁶ cells/mL;
   perfusing the culture at one or more perfusion rates of less than or equal to 0.5 culture volumes/day (V/d) until the culture reaches a desired target criteria;
   increasing the volume of the culture medium to about 70% to less than or equal to 100% of the final bioreactor working volume by differential perfusion wherein the feed rate is greater than the permeate rate; and
   perfusing the culture at one or more perfusion rates of less than or equal to 0.5 culture volumes/day (V/d) until the culture is terminated or harvested.
E41. A method for producing an isolated, purified, recombinant protein comprising
   initiating a culture in a bioreactor with a culture volume that is at least 50% of the final bioreactor working volume;
   inoculating the culture with cells expressing the protein at a cell density of at least 6×10⁶ cells/mL;
   maintaining the culture in a growth phase by perfusing the culture at a rate of at least 0.05 V/d until the culture reaches a desired target criteria;
   increasing the culture volume by differential perfusion until it is at least 70% of the final bioreactor working volume; and
   maintaining the culture in a production phase by perfusing the culture at a rate of at least 0.05 V/d;
   harvesting the recombinant protein;
   processing the recombinant protein through one or more unit operations; and
   obtaining an isolated, purified, recombinant protein.
E42. The method according to E41, wherein at least one unit operation is an affinity chromatography unit operation.
E43. The method according to E42, wherein the affinity chromatography medium is selected from Protein A or immobilized metal affinity chromatography (IMAC).
E44. The method according to E41, wherein at least one unit operation is a polish chromatography unit operation.
E45. The method according to E44, wherein the polish chromatography medium is selected from anion exchange chromatography, cation exchange chromatography, multi-modal chromatography, hydrophobic interaction chromatography, and hydroxyapatite chromatography.
E46. The method according to E45, wherein at least one polish chromatography medium is operated in bind and elute mode, flow through mode, or frontal mode.
E47. The method according to E41, wherein at least one unit operation is selected from virus inactivation, virus filtration, depth filtration, and UF/DF.
E48. A method for producing a recombinant protein comprising
   initiating a culture in a bioreactor with cells engineered to express the protein;
   culturing the cells by perfusing the culture at a rate of less than or equal to 0.5 V/d to maintain a culture volume of at least 60% of the final bioreactor working volume;
   subjecting the culture to a feed up phase to increase the culture volume to less than or equal to 100% of the final bioreactor working volume at least 24 hours post-inoculation;
   following the feed up phase by perfusing the culture at a rate of less than or equal to 0.5 V/d to maintain the culture volume greater than 70% of the final bioreactor working volume; and
   harvesting the recombinant protein.

Additional non-limiting example embodiments/features include:
F1. A method for culturing cells to produce a recombinant protein, comprising:
   initiating a culture in a bioreactor at a culture volume that is at least 50% of the final bioreactor working volume;
   inoculating the culture with cells engineered to express the recombinant protein;
   perfusing the culture at one or more perfusion rates of less than or equal to 0.5 culture volumes/day (V/d) until the culture reaches one or more desired target criteria;
   increasing the culture volume to a final culture volume; and
   once the final culture volume is achieved, perfusing the culture at one or more perfusion rates of less than or equal to 0.5 culture volumes/day (V/d) until the culture is terminated or harvested.
F2. The method according to F1, wherein the culture is initiated at a culture volume that is 50% to 75% of the final bioreactor working volume.
F3. The method according to F1 or F2, wherein the culture is initiated at a culture volume that is 60% to 70% of the final bioreactor working volume.
F4. The method according to any one of F1 to F3, wherein the culture is inoculated at a cell density of 5×10⁶ cells/mL to 50×10⁶ cells/mL.
F5. The method according to any one of F1 to F4, wherein the culture is inoculated at a cell density of 6×10⁶ cells/mL to 20×10⁶ cells/mL.
F6. The method according to any one of F1 to F5, wherein the culture is inoculated at a cell density of 6×10⁶ cells/mL to 12×10⁶ cells/mL.
F7. The method according to any one of F1 to F6, wherein the bioreactor is operated in batch mode for up to 24 hours following inoculation.
F8. The method according to any one of F1 to F7, wherein the culture is in a growth phase prior to the increase in culture volume to the final culture volume.
F9. The method according to F8, wherein the duration of the growth phase is less than or equal to 60% of the culture duration.
F10. The method according to F8 or F9, wherein the culture temperature during the growth phase is 35° C. to 37° C.
F11. The method according to any one of F1 to F10, wherein the culture is in a production phase following the increase in culture volume to the final culture volume.
F12. The method according to F11, wherein the culture temperature during the production phase is 28° C. to 35° C.
F13. The method according to F11 or F12, wherein the duration of the production phase is 10 days to 20 days.
F14. The method according to any one of F11 to F13, wherein the cell density at the start of the production phase is at least 2 times the inoculation cell density.
F15. The method according to any one of F11 to F14, wherein the cell density at the start of the production phase is at least 200×10⁵ cells/mL.
F16. The method according to any one of F1 to F15, wherein:
   the culture is perfused at one or more perfusion rates of 0.05 V/d to 0.5 V/d until the culture reaches one or more desired target criteria, wherein the feed rate(s) and the contemporaneous permeate rate(s) are the same; and/or
   once the final culture volume is achieved, the culture is perfused at one or more perfusion rates of 0.05 V/d to 0.5 V/d until the culture is terminated or harvested, wherein the feed rate(s) and the contemporaneous permeate rate(s) are the same.
F17. The method according to any one of F1 to F16, wherein:
   the culture is perfused at one or more perfusion rates of 0.10 V/d to 0.25 V/d until the culture reaches one or more desired target criteria, wherein the feed rate(s) and the contemporaneous permeate rate(s) are the same; and/or
   once the final culture volume is achieved, the culture is perfused at one or more perfusion rates of 0.10 V/d to 0.25 V/d until the culture is terminated or harvested, wherein the feed rate(s) and the contemporaneous permeate rate(s) are the same.
F18. The method according to any one of F1 to F17, wherein the culture is perfused at a constant perfusion rate prior to increasing the culture volume.
F19. The method according to any one of F1 to F18, wherein, once the final culture volume is achieved, the culture is perfused at a constant perfusion rate until the culture is terminated or harvested.
F20. The method according to any one of F1 to F19, wherein:
   the culture is perfused at a first constant perfusion rate prior to increasing the culture volume; and
   once the final culture volume is achieved, the culture is perfused at a second constant perfusion rate until the culture is terminated or harvested,
   wherein the first constant perfusion rate and the second constant perfusion rate are the same.
F21. The method according to any one of F1 to F20, wherein the one or more desired target criteria are selected from culture volume, final culture volume, bioreactor working volume, final bioreactor working volume, time point, titer, cell density, packed cell volume, product attribute, process, production schedule, plant schedule, and combinations of any of the foregoing.
F22. The method according to any one of F1 to F21, wherein the one or more desired target criteria is a cell density of 100×10⁵ cells/mL to 350×10⁵ cells/mL.
F23. The method according to any one of F1 to F22, wherein the one or more desired target criteria is time post-inoculation, wherein the time post-inoculation is 24 hours to 72 hours.
F24. The method according to any one of F1 to F23, wherein the culture volume is increased to the final culture volume using differential perfusion, wherein the differential perfusion comprises one or more feed rates and one or more permeate rates.
F25. The method according to F24, wherein at least one of the one or more feed rates is less than or equal to 0.50 V/d and at least one of the one or more permeate rates is less than or equal to 0.20 V/d, wherein each of the one or more feed rates is greater than the contemporaneous permeate rate.
F26. The method according to F24 or F25, wherein at least one of the one or more feed rates is less than or equal to 0.40 V/d and at least one of the one or more permeate rates is less than or equal to 0.15 V/d, wherein each of the one or more feed rates is greater than the contemporaneous permeate rate.
F27. The method according to F24, wherein each of the one or more feed rates is less than or equal to 0.50 V/d and each of the one or more permeate rates is less than or equal to 0.20 V/d, wherein each of the one or more feed rates is greater than the contemporaneous permeate rate.
F28. The method according to F24 or F25, wherein each of the one or more feed rates is less than or equal to 0.40 V/d and each of the one or more permeate rates is less than or equal to 0.15 V/d, wherein each of the one or more feed rates is greater than the contemporaneous permeate rate.
F29. The method according to any one of F24 to F28, wherein the one or more feed rates and the one or more permeate rates are selected to increase the media volume in the bioreactor to a volume of less than or equal to 100% of the bioreactor working volume in the time needed for at least one population doubling of the culture.
F30. The method according to any one of F24 to F29, wherein the differential perfusion comprises a constant feed rate and a constant permeate rate, wherein the constant feed rate is greater than the constant permeate rate.
F31. The method according to any one of F24 to F30, wherein the differential perfusion continues until the culture volume in the bioreactor is 70% to 100% of the final bioreactor working volume.
F32. The method according to any one of F1 to F31, wherein the final working volume of the bioreactor is 70% to 100% of the bioreactor volume.
F33. A method for culturing cells to produce a recombinant protein, comprising:
   initiating a culture in a bioreactor at a culture volume that is 50% to less than 70% of the final bioreactor working volume;
   inoculating the culture with cells engineered to express the recombinant protein, wherein the culture is inoculated at a cell density of 5×10⁶ cells/mL to 50×10⁶ cells/mL;
   operating the bioreactor in batch mode for up to 24 hours following inoculation;
   perfusing the culture at one or more perfusion rates of less than or equal to 0.5 culture volumes/day (V/d) for 24 hours to 72 hours, wherein the feed rate(s) and the contemporaneous permeate rate(s) are the same;
   increasing the culture volume to 70% to 100% of the final bioreactor working volume; and
   once the final culture volume is achieved, perfusing the culture at one or more perfusion rates of less than or equal to 0.5 culture volumes/day (V/d) until the culture is terminated or harvested, wherein the feed rate(s) and the contemporaneous permeate rate(s) are the same.
F34. The method according to F33, wherein the culture is inoculated at a cell density of 6×10⁶ cells/mL to 12×10⁶ cells/mL.
F35. The method according to F33 or F34, wherein the culture is perfused at one or more perfusion rates of 0.05 V/d to 0.5 V/d for 24 hours to 60 hours prior to increasing the culture volume.
F36. The method according to any one of F33 to F35, wherein the culture is perfused at one or more perfusion rates of 0.05 V/d to 0.5 V/d for 24 hours to 48 hours prior to increasing the culture volume.
F37. The method according to any one of F33 to F36, wherein the culture is perfused at one or more perfusion rates of 0.05 V/d to 0.5 V/d for 48 hours prior to increasing the culture volume.
F38. The method according to F33 or F34, wherein the culture is perfused at one or more perfusion rates of 0.10 V/d to 0.25 V/d for 24 hours to 60 hours prior to increasing the culture volume.
F39. The method according to any one of F33, F34, or F38, wherein the culture is perfused at one or more perfusion rates of 0.10 V/d to 0.25 V/d for 24 hours to 48 hours prior to increasing the culture volume.
F40. The method according to any one of F33, F34, F38, or F39, wherein the culture is perfused at one or more perfusion rates of 0.10 V/d to 0.25 V/d for 48 hours prior to increasing the culture volume.
F41. The method according to any one of F33 to F40, wherein the culture is perfused at a constant perfusion rate prior to increasing the culture volume.
F42. The method according to any one of F33 to F41, wherein, once the final culture volume is achieved, the culture is perfused at one or more perfusion rates of 0.05 V/d to 0.5 V/d until the culture is terminated or harvested.
F43. The method according to any one of F33 to F42, wherein, once the final culture volume is achieved, the culture is perfused at one or more perfusion rates of 0.10 V/d to 0.25 V/d until the culture is terminated or harvested.
F44. The method according to any one of F33 to F43, wherein, once the final culture volume is achieved, the culture is perfused at a constant perfusion rate until the culture is terminated or harvested.
F45. The method according to any one of F33 to F44, wherein:
   the culture is perfused at a first constant perfusion rate prior to increasing the culture volume; and
   once the final culture volume is achieved, the culture is perfused at a second constant perfusion rate until the culture is terminated or harvested,
   wherein the first constant perfusion rate and the second constant perfusion rate are the same.
F46. The method according to any one of F33 to F45, wherein the culture volume is increased to the final culture volume using differential perfusion.
F47. A method for culturing cells to produce a recombinant protein, comprising:
   initiating a culture in a bioreactor at a culture volume that is 50% to less than 70% of the final bioreactor working volume;
   inoculating the culture with cells engineered to express the recombinant protein, wherein the culture is inoculated at a cell density of 5×10⁶ cells/mL to 50×10⁶ cells/mL;
   operating the bioreactor in batch mode for up to 24 hours following inoculation;
   perfusing the culture at one or more perfusion rates of less than or equal to 0.5 culture volumes/day (V/d) until the cell density reaches a value in the range of 100×10⁵ cells/mL to 350×10⁵ cells/mL, wherein the feed rate(s) and the contemporaneous permeate rate(s) are the same;
   increasing the culture volume to 70% to 100% of the final bioreactor working volume; and
   once the final culture volume is achieved, perfusing the culture at one or more perfusion rates of less than or equal to 0.5 culture volumes/day (V/d) until the culture is terminated or harvested, wherein the feed rate(s) and the contemporaneous permeate rate(s) are the same.
F48. The method according to F47, wherein the culture is inoculated at a cell density of 6×10⁶ cells/mL to 12×10⁶ cells/mL.
F49. The method according to F47 or F48, wherein the culture is perfused at one or more perfusion rates of 0.05 V/d to 0.5 V/d until the cell density reaches a value in the range of 100×10⁵ cells/mL to 350×10⁵ cells/mL.
F50. The method according to any one of F47 to F49, wherein the culture is perfused at one or more perfusion rates of 0.10 V/d to 0.25 V/d until the cell density reaches a value in the range of 100×10⁵ cells/mL to 350×10⁵ cells/mL.
F51. The method according to any one of F47 to F50, wherein the culture is perfused at a constant perfusion rate until the cell density reaches a value in the range of 100×10⁵ cells/mL to 350×10⁵ cells/mL.
F52. The method according to any one of F47 to F51, wherein, once the final culture volume is achieved, the culture is perfused at one or more perfusion rates of 0.05 V/d to 0.5 V/d until the culture is terminated or harvested.
F53. The method according to any one of F47 to F52, wherein, once the final culture volume is achieved, the culture is perfused at one or more perfusion rates of 0.10 V/d to 0.25 V/d until the culture is terminated or harvested.
F54. The method according to any one of F47 to F53, wherein, once the final culture volume is achieved, the culture is perfused at a constant perfusion rate until the culture is terminated or harvested.
F55. The method according to any one of F47 to F54, wherein:
   the culture is perfused at a first constant perfusion rate prior to increasing the culture volume; and
   once the final culture volume is achieved, the culture is perfused at a second constant perfusion rate until the culture is terminated or harvested,
   wherein the first constant perfusion rate and the second constant perfusion rate are the same.
F56. The method according to any one of F47 to F55, wherein the culture volume is increased to the final culture volume using differential perfusion.
F57. The method according to any one of F1 to F56, wherein the bioreactor is connected to a separation system.
F58. The method according to F57, wherein the separation system is a recirculating tangential flow filter system or an alternating tangential flow filtration system.
F59. The method according to any one of F1 to F58, wherein the volume of the bioreactor is 200 liters to 20,000 liters.
F60. The method according to any one of F1 to F59, wherein the bioreactor is a single use bioreactor or a stainless steel bioreactor.
F61. The method according to any one of F1 to F60, further comprising:
   harvesting the recombinant protein;
   processing the recombinant protein through one or more unit operations; and obtaining an isolated, purified recombinant protein.
F62. The method according to F61, wherein at least one of the one or more unit operations is an affinity chromatography unit operation.
F63. The method according to F61 or F62, wherein at least one of the one or more unit operations is a polishing chromatography unit operation.
F64. The method according to any one of F61 to F63, wherein at least one of the one or more unit operations is selected from virus inactivation, virus filtration, depth filtration, and ultrafiltration/diafiltration (UF/DF).
F65. The method according to any one of F1 to F64, wherein the recombinant protein is an antibody.
F66. The method according to any one of F1 to F64, wherein the recombinant protein is an IgG antibody.
F67. The method according to any one of F1 to F64, wherein the recombinant protein is an antibody fragment.
F68. The method according to any one of F1 to F67, wherein the cells are CHO cells.

### BRIEF DESCRIPTION OF THE DRAWINGS

FIG. 1 shows an offset schematic representation for a non-limiting example lean perfusion cell culture from culture initiation to harvest which highlights changes in the culture volume, as well as the feed and permeate rates. At the initiation of the culture and during the growth phase, the culture volume is kept low, at least 50% to 75% of the final working volume of the bioreactor. Maintaining the feed and permeate at the same rate keeps the culture volume static. During the feed up period, the feed rate is increased compared to the permeate rate, resulting in an increase in the culture volume to a desired final volume (typically greater than or equal to 75% to 100% of the final working volume), after which the feed and the permeate are maintained at the same rate to maintain a static culture volume. The top plot shows the change in culture volume from initiation to harvest. The middle and bottom plots show that the feed rate is maintained at the same rate as the permeate rate (Rates 1 and 3), except during the feed up phase. During the feed up phase, the feed rate (Rate 2) is increased to facilitate an increase in the culture volume in the bioreactor.

FIG. 2 shows a schematic representation of culture volume change over the course of the culture from initiation to harvest. A typical lean perfusion culture with a single feed up phase (black line) is compared to a typical fed batch culture with multiple feeds (gray line). Both cultures are initiated at a low culture volume, at least 50% to 75% of the bioreactor final working volume. The lean perfusion method makes use of a shortened growth phase at a low culture volume followed by a transition to the production phase with a feed up that increases the culture volume, maximizing the bioreactor working volume during both phases. The fed batch method slowly increases the culture volume by means of stepwise or intermittent feeds, achieving the final working volume at a desired cell density as the culture begins to decline.

FIG. 3 shows the average viable cell density (VCD) of the lean perfusion cultures and the fed batch cultures. Lean perfusion (LP): mAb 1 solid dark gray line, mAb 2 solid black line, mAb 3 solid light gray line. Fed batch (FB): mAb 1 dashed dark gray line, mAb 2 dashed black line, mAb 3 dashed light gray line.

FIGS. 4A, 4B, and 4C show the average specific productivity (pg/cell/day) of the lean perfusion cultures and the fed batch cultures. Lean perfusion (LP) solid black column, fed batch (FB) solid gray column. mAb 1 (FIG. 4A), mAb 2 (FIG. 4B), mAb 3 (FIG. 4C).

FIGS. 5A, 5B, and 5C show the average final day titer of the lean perfusion cultures and the fed batch cultures. Lean perfusion (LP) solid black column, fed batch (FB) solid gray column. mAb 1 (FIG. 5A), mAb 2 (FIG. 5B), mAb 3 (FIG. 5C).

FIG. 6 shows average lactate production during the lean perfusion cultures and the fed batch cultures. Lean perfusion culture: mAb 1 solid dark gray line, mAb 2 solid black line, mAb 3 solid light gray line. Fed batch: mAb 1 dashed dark gray line, mAb 2 dashed black line, mAb 3 dashed light gray line.

FIG. 7 shows a schematic representation of the perfusion rates over the course of a culture from initiation to harvest. A typical lean perfusion culture (black line) maintains a low perfusion rate (<0.5 V/d) for the entire duration of the culture, whereas a typical perfusion culture (gray line) may begin at the desired final perfusion rate (typically 1.0 V/d or higher) or optionally begin at a lower rate, increasing to the final rate early in the culture.

FIG. 8 shows a schematic of the feed media used over the duration of the culture from initiation to harvest. Lean perfusion culture (black line, low perfusion rate); perfusion culture (gray line, higher perfusion rate).

FIG. 9 shows the magnitude of the feed medium volume used during a culture. Lean perfusion culture (LP, black column), perfusion culture (PC, dark gray column).

FIG. 10 shows a schematic representation of the change in culture volume over time. The increasing culture volume during lean perfusion culture (black line) compared to the static culture volume for the perfusion culture (gray line).

FIG. 11 shows the average viable cell density (VCD) of the lean perfusion cultures compared to the perfusion cultures. Lean perfusion cultures: mAb 2 (solid black line), mAb 3 (solid gray line). Perfusion cultures: mAb 2 (dashed black line), mAb 3 (dashed gray line).

FIGS. 12A and 12B show the average final day titer of the lean perfusion culture compared to the perfusion culture. Lean perfusion culture (LP): black column, perfusion culture (PR): gray column. mAb 2 (FIG. 12A), mAb 3 (FIG. 12B).

FIGS. 13A and 13B show the average specific productivity (pg/cell/day) of the lean perfusion cultures compared to the perfusion cultures. Lean perfusion culture (LP): solid black column, perfusion culture (PR): solid gray column. mAb 2 (FIG. 13A), mAb 3 (FIG. 13B).

FIGS. 14A and 14B show the average packed cell volume (%) of the lean perfusion cultures compared to the perfusion cultures. Lean perfusion culture (LP): solid black column, perfusion culture (PR): solid gray column. mAb 2 (FIG. 14A), mAb 3 (FIG. 14B).

FIG. 15 shows the average viable cell densities (VCD) achieved by mAb 4 lean perfusion cultures (solid black line) and fed batch cultures (solid gray line) from initiation to harvest.

FIG. 16 shows the average final day titer (g/L) for mAb 4 lean perfusion cultures (black column) and fed batch cultures (gray column).

FIG. 17 shows a comparison of average lactate concentration for mAb 4 lean perfusion cultures (solid black line) and fed batch cultures (solid gray line) from initiation to harvest.

### DETAILED DESCRIPTION

Described herein is a lean perfusion cell culture method to produce recombinant protein products of interest. Lean perfusion is a surprisingly efficient and productive culture method compared to traditional batch, fed batch, and perfusion culture methods. The method makes use of perfusion, or media exchange, to add fresh media to and remove spent medium from the culture. The perfusion rate throughout the culture, from initiation to harvest, is maintained at less than or equal to 50% of the perfusion rate used in a typical perfusion culture method, which is operated at a high perfusion rate. In addition, lean perfusion cultures are initiated at high cell density in a low culture volume, at least 50% of the final working volume of the bioreactor. This allows for more efficient use of the bioreactor working volume during the growth phase. Once one or more desired target criteria are met, the culture volume is then increased and maintained throughout the production phase until harvest. With an increased culture volume and relatively lower cell density, the lean perfusion culture makes efficient use of the full bioreactor working volume during the production phase, which, combined with the low perfusion rate, reduces feed media use and waste generation. Compared to high perfusion rate perfusion processes, these features also allow for more flexibility in bioreactor type and size (e.g., single use and/or large-scale stainless-steel bioreactors, at capacities from hundreds of liters, to up to tens of thousands of liters) as well as the use of common separation systems. For example, the lean perfusion method enables the combination of single use separation systems, such as alternating tangential flow systems, with large scale (greater than 5,000 L) stainless-steel bioreactors.

As used herein, “bioreactor volume” refers to the actual vessel size of the bioreactor, for example, a 2,000 L single use bioreactor or a 20,000 L stainless steel bioreactor. The bioreactor volume is divided into the working volume space and the headspace. As used herein, “working volume” or “bioreactor working volume” refers to the volume within the bioreactor in which the cell culture is operated and is typically expressed as a percentage of the bioreactor volume. For example, a 2,000 L stirred tank single use bioreactor may have a working volume of 100% of the bioreactor volume. For stainless steel bioreactors, the working volume is typically up to about 90% of the bioreactor volume. Some cell culture operations, including the lean perfusion culture processes described herein, make use of different percentages of the available working volume over the duration of the culture. For example, fed batch and lean perfusion cultures are initiated at a culture volume that is 50% or more of the final bioreactor working volume and as the culture progresses, the culture volume is increased to as much as 100% of the final working volume of the bioreactor. “Final working volume,” as used herein, refers to the greatest volume within bioreactor in which the cell culture is operated, typically the volume during the production phase. As used herein, “culture volume” refers to the volume of all culture components that are in the bioreactor and attached equipment and associated flow paths, including the culture medium, cells, cell debris, bubbles, and foam. The culture volume is expressed as a percentage of the final working volume. For perfusion cultures, including lean perfusion cultures, the culture volume encompasses the working volume as well as the holdup volume in the perfusion flow path, including filters. For large scale single use (SU) bioreactor cultures, e.g., cultures greater than 500 L, the holdup volume is negligible. Both the working volume and the culture volume may be increased over the culture duration, depending on the process design.

Batch cell culture is executed in a closed bioreactor system that contains a fixed volume of a culture medium, which serves as the source of nutrients to support the culture over its duration. Since no additional media will be added, a typical batch culture begins with a culture medium containing all essential nutrient components added at or near 100% of the bioreactor working volume. Large-scale bioreactors operating with volumes up to tens of thousands of liters are favored. The culture is inoculated at a low cell density with cells in the growth phase to maintain waste product levels below a threshold that would impact cell culture viability and productivity for as long as possible. As used herein, “cell density” refers to the number of living cells in a given volume of culture medium. Harvest typically begins when the product quality, cell viability, and/or productivity declines.

Fed batch culture is the most commonly used mammalian cell culture method for producing recombinant proteins. The fed batch strategy is flexible and accommodates a wide range of bioreactor types and sizes and can be scaled up or down to meet supply demands. The fed batch culture offers an improvement over batch culture methods by supplementing the culture with fresh nutrient media to replenish depleted nutrients during the course of the culture, but like batch culture, the spent medium containing waste products and the like is not removed from the bioreactor and accumulates over time. Like batch cultures, large-scale bioreactors operating at volumes up to tens of thousands of liters are favored for fed batch operations due to low titers and to meet product demands. Harvest typically begins when the product quality, cell viability, and/or productivity of the cells decline(s).

A typical fed-batch culture is initiated into a basal culture medium containing essential nutrients at a culture volume that is at least 50% of the final working volume of the bioreactor. Fed batch cultures are initiated in the growth phase; the culture is inoculated at a low cell density (such as, e.g., (2−60)×10⁵ cells/mL) to maintain waste product levels below a threshold that would impact cell culture health and viability for as long as possible. As the cell concentration increases, supplemental nutrient media are added to the bioreactor, up to 100% of the final working volume of the bioreactor, to prolong cell life and improve productivity. These media supplements can occur at regular intervals or in a stepwise manner during the course of the culture. Like with batch cultures, cells, proteins, spent culture media, product- and process-related impurities, waste products, and the like are all retained and accumulate in the bioreactor. With time, these components build up and the culture health begins to decline, which negatively impacts cell mass and titer. A fed batch culture is typically maintained for 8 to 15 days. Harvest typically begins when the product quality, cell viability, and/or productivity of the cells decline(s).

In recent years, perfusion or media exchange has gained popularity for use in mammalian cell culture processes. As with fed batch operations, nutrient solutions and/or various media formulations are fed into the bioreactor over the course of the culture. Unlike batch and fed batch methods, spent culture media containing waste products and the like are removed in the permeate during the culture. Perfusion cultures make use of separation systems comprising pumps used to direct the contents of the bioreactor through the separation systems. Typically, the separation systems comprise one or more membrane filters (often operated in tangential flow mode) that selectively retain or return cell culture components back to the bioreactor. Spent culture media, waste products and impurities, and other components, including recombinant proteins, can be selectively removed in the permeate depending on the selection criteria of the separation system. For membrane filters, pore size or molecular weight cut-off of the filter(s) are used. The permeate can be sent to waste or collected for harvest. In general, perfusion cultures are initiated and maintained at or near 100% of the working volume of the bioreactor, and the cultures achieve high cell densities and/or packed cell volumes due to the continuous exchange of media.

As used herein, “packed cell volume” (PCV), also referred to as “percent packed cell volume” (% PCV), is the ratio of the volume occupied by the cells, to the total volume of cell culture (see Stealer, et al., (2006) Biotechnol. Bioeng. December 20:95(6):1228-33). Packed cell volume is a function of cell density and cell diameter; increases in packed cell volume could arise from increases in cell density, cell diameter, or both. Packed cell volume is a measure of the solids content in the cell culture. In general, a cell culture with higher solids content will require more processing to separate the solid material from the desired product during harvest and downstream purification steps. Also, the desired product can become trapped in the solids and lost during the harvest process, resulting in a decreased product yield. Since host cells vary in size and cell cultures also contain dead and dying cells and other cellular debris, packed cell volume is a more accurate way to describe the solids content within a cell culture than cell density or viable cell density. In addition, some cells, when in a growth-arrested state, will increase in size, so the packed cell volume prior to growth-arrest and post growth-arrest will likely differ, due to an increase in biomass as a result of cell size increase.

Like batch and fed batch cultures, in perfusion cultures, the cells are typically retained in the bioreactor for the duration of the culture. Unlike batch and fed batch cultures, perfusion cultures typically have a higher cell mass, which in turn produces a larger amount of waste and byproducts over the course of the culture compared to a batch or fed batch process, while also requiring large volumes of fresh media replenishment. Cell densities achieved during a perfusion culture are typically (20−120)×10⁶ cells/mL or higher. Unlike batch and fed batch cultures, perfusion cultures are performed in low volume bioreactors (typically 2,000 L or less) due to the limits of the separation systems that enable continuous media exchange and the high consumption of media. These separation systems are sensitive to the high cell densities and high flow rates common with typical perfusion cultures. Moreover, the high packed cell volumes that can be achieved during a perfusion culture, even in a low volume bioreactor, generally limit harvest options.

Perfusion cultures are based on media exchange, i.e., the addition of fresh culture media (feed media) to the bioreactor and removal of a similar volume of spent culture media, as permeate, through the separation system(s). Fresh cell culture media may be added to the bioreactor in a stepwise, intermittent, and/or continuous manner. Spent culture media and selected waste products are removed from the bioreactor using one or more separation systems and then exit in the permeate. A conventional perfusion culture is operated at a high perfusion rate, with 1.0 V/d or more of fresh media added to the bioreactor and an equal volume of spent media removed. In some cases, perfusion cultures are started at a perfusion rate of 0.5 V/d or higher and quickly increase in rate. This high perfusion rate consumes a greater quantity of feed media than an equivalent sized fed batch culture and also produces an equal volume of spent media that must be disposed of. Since the waste and byproducts do not accumulate in the bioreactor to the degree that they do in batch and fed batch cultures, these cultures may be maintained for a longer period, for example, 15 to 90 days or more.

Perfusion cultures make use of separation systems for media exchange. The filters and/or pumps of conventional separation systems can be negatively impacted by the high flow rates, high cell densities, high protein titers, and/or high volumes of waste and byproducts that are associated with these cultures. As such, perfusion cultures are typically performed in smaller scale bioreactors, e.g., at least 200 L, typically 1,000 L to 2,000 L, to minimize the impact on the separation systems. In addition, packed cell volumes can be much higher in these cultures, which limits the ability to remove all the desired protein from the bioreactor, resulting in lower harvest titers.

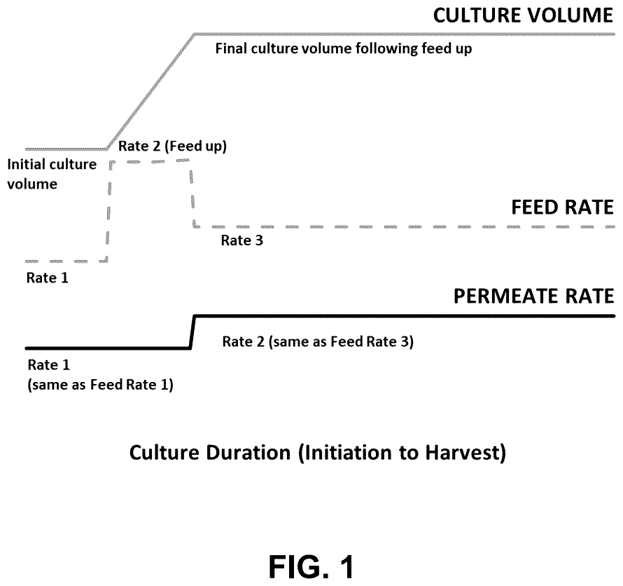

The “lean perfusion” or “lean perfusion rate” cell culture method described herein is unique. Like a fed batch culture, a lean perfusion cell culture is initiated at a low culture volume (between 50% to 75% of the final bioreactor working volume); however, unlike a fed batch culture, a lean perfusion culture is inoculated at a high cell density. The lean perfusion method is similar to a typical perfusion culture in that it makes use of media exchange; however, it differs in that the perfusion rate is maintained at or below 0.5 V/d throughout the entire process (except potentially the feed up stage). The perfusion rate is maintained at a level that is sufficient to ensure removal of waste byproducts to avoid culture toxicity while delivering sufficient nutrients via the fresh media to maintain essential cellular functions. Unlike both fed batch and perfusion cultures, the lean perfusion culture maintains a lower cell density in a higher culture volume (at or below 100% of the final bioreactor working volume) during the production phase, which again reduces the amount of feed needed to maintain the culture and the amount of waste in the permeate flow (FIG. 1).

By initiating and maintaining a low culture volume with higher cell density during the growth phase and transitioning to a higher culture volume with lower cell density prior to the production phase, the lean perfusion method makes efficient use of the working volume of the bioreactor at each culture phase which, in combination with the low perfusion rates, optimizes the amount of culture media used during each phase and reduces the volume of spent media discarded, as compared to fed batch and perfusion cultures. In addition, maintaining a lower cell density and/or packed cell volume during the production phase reduces the amount of waste that must be processed during harvest and downstream purification. The lean perfusion method provides flexibility in the type and scale of bioreactor that can be used, enabling the use of anything from single use bioreactors to large capacity stainless steel bioreactors. In addition, the lower cell densities, lower packed cell volumes, and decreased spent media volumes generated during the production phase make it possible to use common separation systems, such as single use separation systems, for perfusion in combination with larger scale stainless steel bioreactors (up to 10,000 L to 20,000 L or more) and permit more harvest flexibility.

The lean perfusion method is also advantageous in that inoculating a low volume cell culture at a high cell density reduces the duration of the growth phase, quickly achieving a desired target criteria such as cell density as well as reducing the amount of feed media required, compared to typical fed batch and perfusion culture methods. Packed cell volume during the production phase was reduced by up to 50%, compared to a typical perfusion culture method. The specific productivity of the lean perfusion culture increased 22% to 51% as compared to a typical fed batch culture method. Final titers showed an increase of 3 times or more as compared to a typical fed batch method and produced equivalent or higher final titers and specific productivity compared to a typical perfusion culture method. In addition, the lean perfusion method maintained low production of inhibitory metabolic byproducts, such as lactic acid, through the use of low feed and permeate rates while increasing cell mass, as compared to a typical fed batch method. The lean perfusion method maintained equivalent or improved performance while overcoming limitations of fed batch and perfusion culture methods.

The lean perfusion culture is initiated in a bioreactor with a cell culture medium suitable for the culture. In one embodiment, the cell culture medium is a basal medium containing essential nutrients. In one embodiment, the culture volume in the bioreactor at culture initiation is at least 50% of the final bioreactor working volume. In one embodiment, the culture volume is at least about 50% to at least about 75% of the final bioreactor working volume. In one embodiment, the culture volume is at least about 50% to at least about 70% of the final bioreactor working volume. In one embodiment, the culture volume is at least about 50% to at least about 65% of the final bioreactor working volume. In one embodiment, the culture volume is at least about 50% to at least about 60% of the final bioreactor working volume. In one embodiment, the culture volume is at least about 50% to at least about 55% of the final bioreactor working volume. In one embodiment, the culture volume is at least about 55% to at least about 75% of the final bioreactor working volume. In one embodiment, the culture volume is at least about 55% to at least about 70% of the final bioreactor working volume. In one embodiment, the culture volume is at least about 55% to at least about 65% of the final bioreactor working volume. In one embodiment, the culture volume is at least about 55% to at least about 60% of the final bioreactor working volume. In one embodiment, the culture volume is at least about 70% to at least about 75% of the final bioreactor working volume. In one embodiment, the culture volume is at least about 60% to at least about 70% of the final bioreactor working volume. In one embodiment, the culture volume is at least about 60% to at least about 65% of the final bioreactor working volume. In one embodiment, the culture volume is at least about 65% to at least about 75% of the final bioreactor working volume. In one embodiment, the culture volume is at least about 70% to at least about 75% of the final bioreactor working volume.

In one embodiment, the culture volume is at least 50%, 55%, 60%, 65%, 66%, 67%, 68%, 69%, 70%, 75%, 80%, 85%, 90%, or 95% or more of the final bioreactor working volume. In one embodiment, the culture volume is greater than 50% of the final bioreactor working volume. In one embodiment, the culture volume is greater than 55% of the final bioreactor working volume. In one embodiment, the culture volume is greater than 60% of the final bioreactor working volume. In one embodiment, the culture volume is greater than 65% of the final bioreactor working volume. In one embodiment, the culture volume is greater than 70% of the final bioreactor working volume. In one embodiment, the culture volume is greater than 75% of the final bioreactor working volume. In one embodiment, the culture volume is at least 66% of the final bioreactor working volume. In one embodiment, the culture volume is at least 67% of the final bioreactor working volume. In one embodiment, the culture volume is at least 68% of the final bioreactor working volume. In one embodiment, the culture volume is at least 69% of the final bioreactor working volume. In one embodiment, the culture volume is at least about 70% of the final bioreactor working volume. In one embodiment, the culture volume is at least 75% of the final bioreactor working volume.

In one embodiment, the culture volume in the bioreactor at culture initiation is about 50% to about 75% of the final bioreactor working volume. In one embodiment, the culture volume in the bioreactor at culture initiation is about 50% to about 70% of the final bioreactor working volume. In one embodiment, the culture volume in the bioreactor at culture initiation is about 50% to about 65% of the final bioreactor working volume. In one embodiment, the culture volume in the bioreactor at culture initiation is about 50% to about 60% of the final bioreactor working volume. In one embodiment, the culture volume in the bioreactor at culture initiation is about 50% to about 55% of the final bioreactor working volume. In one embodiment, the culture volume in the bioreactor at culture initiation is about 55% to about 75% of the final bioreactor working volume. In one embodiment, the culture volume in the bioreactor at culture initiation is about 55% to about 70% of the final bioreactor working volume. In one embodiment, the culture volume in the bioreactor at culture initiation is about 55% to about 65% of the final bioreactor working volume. In one embodiment, the culture volume in the bioreactor at culture initiation is about 55% to about 60% of the final bioreactor working volume. In one embodiment, the culture volume in the bioreactor at culture initiation is about 70% to about 75% of the final bioreactor working volume. In one embodiment, the culture volume in the bioreactor at culture initiation is about 60% to about 70% of the final bioreactor working volume. In one embodiment, the culture volume in the bioreactor at culture initiation is about 60% to about 65% of the final bioreactor working volume. In one embodiment, the culture volume in the bioreactor at culture initiation is about 65% to about 75% of the final bioreactor working volume. In one embodiment, the culture volume in the bioreactor at culture initiation is about 70% to about 75% of the final bioreactor working volume.

In one embodiment, the culture volume in the bioreactor at culture initiation is about 50%, about 55%, about 60%, about 65%, about 66%, about 67%, about 68%, about 69%, about 70%, about 75%, about 80%, about 85%, about 90%, or about 95% or more of the final bioreactor working volume. In one embodiment, the culture volume in the bioreactor at culture initiation is about 55% of the final bioreactor working volume. In one embodiment, the culture volume in the bioreactor at culture initiation is about 60% of the final bioreactor working volume. In one embodiment, the culture volume in the bioreactor at culture initiation is about 65% of the final bioreactor working volume. In one embodiment, the culture volume in the bioreactor at culture initiation is about 66% of the final bioreactor working volume. In one embodiment, the culture volume in the bioreactor at culture initiation is about 67% of the final bioreactor working volume. In one embodiment, the culture volume in the bioreactor at culture initiation is about 68% of the final bioreactor working volume. In one embodiment, the culture volume in the bioreactor at culture initiation is about 69% of the final bioreactor working volume. In one embodiment, the culture volume in the bioreactor at culture initiation is about 70% of the final bioreactor working volume. In one embodiment, the culture volume in the bioreactor at culture initiation is about 75% of the final bioreactor working volume.

The culture is inoculated with cells engineered to express a desired protein. In one embodiment, a culture volume at inoculation of at least 50% of the final bioreactor working volume allows the use of N−1 seed bioreactors with a broad range of working volumes. In one embodiment, the culture is inoculated at a cell density that will enable the culture to meet one or more desired process, production and/or plant parameters. In one embodiment, the desired parameter is a shortened growth phase. For example, in some embodiments, the culture is inoculated at a cell density of at least 5.0×10⁶ cells/mL. In one embodiment, the cell density is at least 6.0×10⁶ cells/mL to at least 50.0×10⁶ cells/mL. In one embodiment, the cell density is at least 6.0×10⁶ cells/mL to at least 25.0×10⁶ cells/mL. In one embodiment, the cell density is at least 6.0×10⁶ cells/mL to at least 20.0×10⁶ cells/mL. In one embodiment, the cell density is at least 6.0×10⁶ cells/mL to at least 15.0×10⁶ cells/mL. In one embodiment, the cell density is at least 6.0×10⁶ cells/mL to at least 14.0×10⁶ cells/mL. In one embodiment, the cell density is at least 6.0×10⁶ cells/mL to at least 13.0×10⁶ cells/mL. In one embodiment, the cell density is at least 6.0×10⁶ cells/mL to at least 11.0×10⁶ cells/mL. In one embodiment, the cell density is at least 6.0×10⁶ cells/mL to at least 15.0×10⁶ cells/mL. In one embodiment, the cell density is at least 6.0×10⁶ cells/mL to at least 10.0×10⁶ cells/mL.

In one embodiment, the inoculated cell density is about 6×10⁶ cells/mL to about 50×10⁶ cells/mL. In one embodiment, the inoculated cell density is about 6×10⁶ cells/mL to about 25×10⁶ cells/mL. In one embodiment, the inoculated cell density is about 6×10⁶ cells/mL to about 20×10⁶ cells/mL. In one embodiment, the inoculated cell density is about 6×10⁶ cells/mL to about 15×10⁶ cells/mL. In one embodiment, the inoculated cell density is about 6×10⁶ cells/mL to about 14×10⁶ cells/mL. In one embodiment, the inoculated cell density is about 6×10⁶ cells/mL to about 13×10⁶ cells/mL. In one embodiment, the inoculated cell density is about 6×10⁶ cells/mL to about 12×10⁶ cells/mL. In one embodiment, the inoculated cell density is about 6×10⁶ cells/mL to about 11×10⁶ cells/mL. In one embodiment, the inoculated cell density is about 6×10⁶ cells/mL to about 10×10⁶ cells/mL.

In one embodiment, the culture is inoculated at a cell density of at least 5×10⁶, 6×10⁶, 7×10⁶, 8×10⁶, 9×10⁶, 10×10⁶, 11×10⁶, 12×10⁶, 13×10⁶, 14×10⁶, 15×10⁶, 20×10⁶, 25×10⁶, 30×10⁶, 35×10⁶, 40×10⁶, 45×10⁶, 50×10⁶ or more cells/mL. In one embodiment, the cell density is at least 7.0×10⁶ cells/mL. In one embodiment, the cell density is at least 8.0×10⁶ cells/mL. In one embodiment, the cell density is at least 9.0×10⁶ cells/mL. In one embodiment, the cell density is at least 10.0×10⁶ cells/mL. In one embodiment, the cell density is at least 11.0×10⁶ cells/mL. In one embodiment, the cell density is at least 12.0×10⁶ cells/mL. In one embodiment, the cell density is at least 13.0×10⁶ cells/mL. In one embodiment, the cell density is at least 14.0×10⁶ cells/mL. In one embodiment, the cell density is at least 15.0×10⁶ cells/mL. In one embodiment, the cell density is at least 20.0×10⁶ cells/mL.

In one embodiment, the culture is inoculated at a cell density of about 5×10⁶, about 6×10⁶, about 7×10⁶, about 8×10⁶, about 9×10⁶, about 10×10⁶, about 11×10⁶, about 12×10⁶, about 13×10⁶, about 14×10⁶, about 15×10⁶, about 20×10⁶, about 25×10⁶, about 30×10⁶, about 35×10⁶, about 40×10⁶, about 45×10⁶, or about 50×10⁶ cells/mL.

In one embodiment, the inoculated cell density is about 7×10⁶ cells/mL. In one embodiment, the inoculated cell density is about 8×10⁶ cells/mL. In one embodiment, the inoculated cell density is about 9×10⁶ cells/mL. In one embodiment, the inoculated cell density is about 10×10⁶ cells/mL. In one embodiment, the inoculated cell density is about 11×10⁶ cells/mL. In one embodiment, the inoculated cell density is about 12×10⁶ cells/mL. In one embodiment, the inoculated cell density is about 13×10⁶ cells/mL. In one embodiment, the inoculated cell density is about 14×10⁶ cells/mL. In one embodiment, the cell inoculated density is about 15×10⁶ cells/mL. In one embodiment, the inoculated cell density is about 20×10⁶ cells/mL.

In one embodiment, the culture is inoculated with animal cells. Preferably the cells are mammalian cells, such as, e.g., CHO cells.

In one embodiment, the culture is maintained in batch mode for up to 48 hours following initiation to establish the cells in the culture environment. During batch mode, no media is added to or removed from the culture. In one embodiment, batch mode is maintained for less than 12 hours. In one embodiment, batch mode is maintained for at least 12 hours to 24 hours (such as, e.g., about 12.5 hours to about 24 hours). In one embodiment, batch mode is maintained for 24 to 48 hours. In one embodiment, media exchange is started when the culture is initiated.

Cell cultures typically comprise two or more distinct phases, including at least one growth phase and at least one production phase. In between, there may be a transition phase during which culture conditions are manipulated to support the shift between the growth and the production phases.

As used herein, the “growth phase” refers to the period of exponential cell growth (i.e., the log phase) in which the cells are generally rapidly dividing. Culture conditions are established that promote cell proliferation and viability. The length of the growth phase can be based on a variety of factors including, for example, the cell type, the cell density, the rate of cell growth, the culture conditions, production and/or plant scheduling or timelines. The “culture duration,” as used herein, refers to the time from the initiation of the culture until its termination or harvest. The growth phase for a typical fed batch or perfusion rate culture can be up to 75% of the culture duration. The lean perfusion method described herein has a shortened growth phase, up to 60% of the culture duration. In one embodiment, the growth phase is about 60% (e.g., 60%) of the culture duration. In one embodiment, the growth phase is less than 60% of the culture duration. In one embodiment, the growth phase is about 10% to about 60% of the culture duration. In one embodiment, the growth phase is about 10% to about 50% of the culture duration. In one embodiment, the growth phase is about 10% to about 40% of the culture duration. In one embodiment, the growth phase is about 10% to about 30% of the culture duration. In one embodiment, the growth phase is about 10% to about 20% of the culture duration. In one embodiment, the growth phase is about 20% to about 60% of the culture duration. In one embodiment, the growth phase is about 20% to about 50% of the culture duration. In one embodiment, the growth phase is about 20% to about 40% of the culture duration. In one embodiment, the growth phase is about 20% to about 30% of the culture duration. In one embodiment, the growth phase is about 30% to about 60% of the culture duration. In one embodiment, the growth phase is about 30% to about 50% of the culture duration. In one embodiment, the growth phase is about 30% to about 40% of the culture duration. In one embodiment, the growth phase is about 10% of the culture duration. In one embodiment, the growth phase is about 20% of the culture duration. In one embodiment, the growth phase is about 30% of the culture duration. In one embodiment, the growth phase is about 40% of the culture duration. In one embodiment, the growth phase is about 50% of the culture duration. In one embodiment, the growth phase is about 60% of the culture duration. In one embodiment, the growth phase is about 61%, about 62%, about 63%, about 64%, about 65%, about 66%, about 67%, about 68%, or about 69% of the culture duration. In one embodiment, the growth phase is about 10%, about 20%, about 30%, about 40%, about 50%, about 51%, about 52%, about 53%, about 54%, about 55%, about 56%, about 57%, about 58%, about 59%, or about 60% of the culture duration.

In one embodiment, the growth phase is at least 10% to at least 50% of the culture duration. In one embodiment, the growth phase is at least 10% to at least 40% of the culture duration. In one embodiment, the growth phase is at least 10% to at least 30% of the culture duration. In one embodiment, the growth phase is at least 10% to at least 20% of the culture duration. In one embodiment, the growth phase is at least 20% to at least 60% of the culture duration. In one embodiment, the growth phase is at least 20% to at least 50% of the culture duration. In one embodiment, the growth phase is at least 20% to at least 40% of the culture duration. In one embodiment, the growth phase is at least 20% to at least 30% of the culture duration. In one embodiment, the growth phase is at least 30% to at least 60% of the culture duration. In one embodiment, the growth phase is at least 30% to at least 50% of the culture duration. In one embodiment, the growth phase is at least 30% to at least 40% of the culture duration.

In one embodiment, the growth phase is up to 10% of the culture duration. In one embodiment, the growth phase is up to 20% of the culture duration. In one embodiment, the growth phase is up to 30% of the culture duration. In one embodiment, the growth phase is up to 40% of the culture duration. In one embodiment, the growth phase is up to 50% of the culture duration.

In one embodiment, the growth phase is at least 60% of the culture duration. In one embodiment, the growth phase is up to 61%, 62%, 63%, 64%, 65%, 66%, 67%, 68%, or 69% of the culture duration. In one embodiment, the growth phase is at least 10%, 20%, 30%, 40%, 50%, 51%, 52%, 53%, 54%, 55%, 56%, 57%, 58%, or 59% of the culture duration.

The culture volume is maintained by media exchange, simultaneously feeding fresh medium into the bioreactor and removing spent medium from the culture. The fresh cell culture medium may be added to the bioreactor in a stepwise, intermittent, and/or continuous manner. Spent medium may be removed from the bioreactor in a stepwise, intermittent, and/or continuous manner using one or more separation systems. In one embodiment, spent medium exits in the permeate flow.

During the growth and production phases, a static culture volume is maintained by a constant and simultaneous exchange of fresh medium for spent medium. “Media exchange” and “perfusion” are used interchangeably herein and refer to the simultaneous addition of fresh nutrients and/or medium to (“feed”) and removal of spent medium (“permeate”) from the bioreactor. “Feed rate” and “feed flow rate” are used interchangeably herein and refer to the rate at which fresh nutrients and/or medium are added to the bioreactor. “Permeate rate” and “permeate flow rate” are used interchangeably herein and refer to the rate at which spent medium is removed from the bioreactor through the permeate stream of a separation system. “Spent media” and “conditioned media” are used interchangeably herein and collectively refer to any nutrient depleted culture medium, media containing accumulated waste, byproducts, impurities including product-related impurities, and/or process-related impurities, and the like. Spent media may also contain proteins of interest, such as, e.g., recombinant proteins. “Media exchange rate” and “perfusion rate” are used interchangeably herein and refer to the rate at which fresh nutrients and/or medium are added to (“feed rate”) and spent medium is removed from (“permeate rate”) the bioreactor. Typically, the feed rate and permeate rate are the same. In cases where there is a difference in the feed and permeate rates, the perfusion rate would generally be the lower of the two rates.

In one embodiment, during the growth and production phases, the perfusion rate consists of a feed flow and a permeate flow that are maintained at the same rate. In one embodiment, the culture is perfused at one or more feed and perfusion rates during the growth and/or production phases. In one embodiment, the culture is perfused at the same rate(s) during the growth phase and the production phase. In one embodiment, the culture is perfused at a different rate(s) during the growth phase and the production phase.

The media exchange rate or perfusion rate is expressed in terms of a volume over a time period. The volume component is expressed as a portion of the culture volume. The time component is typically expressed in terms of 24 hours or one day. One of skill in the art could easily determine a time period suitable for the production process. As used herein, the media exchange rate or perfusion rate is expressed in terms of a portion of the culture volume per day (V/d). In one embodiment, the perfusion rate is at less than or equal to 0.5 culture volumes/day (V/d). In one embodiment, the perfusion rate is at a rate of at least about 0.05 V/d. In one embodiment, the perfusion rate is at a rate of at least about 0.05 V/d to less than about 0.5 V/d. In one embodiment, the perfusion rate is at a rate of at least about 0.05 V/d to at least about 0.45 V/d. In one embodiment, the perfusion rate is at a rate of at least about 0.05 V/d to at least about 0.40 V/d. In one embodiment, the perfusion rate is at a rate of at least about 0.05 V/d to at least about 0.35 V/d. In one embodiment, the perfusion rate is at a rate of at least about 0.05 V/d to at least about 0.30 V/d. In one embodiment, the perfusion rate is at a rate of at least about 0.05 V/d to at least about 0.25 V/d. In one embodiment, the perfusion rate is at least about 0.05 V/d to at least about 0.20 V/d. In one embodiment, the perfusion rate is at least about 0.05 V/d to at least about 0.15 V/d. In one embodiment, the perfusion rate is at least about 0.05 V/d to at least about 0.10 V/d. In one embodiment, the perfusion rate is at least about 0.1 V/d to about 0.5 V/d.

In one embodiment, the perfusion rate is at least about 0.10 V/d to at least about 0.45 V/d. In one embodiment, the perfusion rate is at least about 0.10 V/d to at least about 0.40 V/d. In one embodiment, the perfusion rate is at least about 0.10 V/d to at least about 0.35 V/d. In one embodiment, the perfusion rate is at least about 0.10 V/d to at least about 0.30 V/d. In one embodiment, the perfusion rate is at least about 0.10 V/d to at least about 0.25 V/d. In one embodiment, the perfusion rate is at least about 0.10 V/d to at least about 0.20 V/d. In one embodiment, the perfusion rate is at least about 0.10 V/d to at least about 0.15 V/d. In one embodiment, the perfusion rate is at least about 0.10 V/d to at least about 0.125 V/d.

In one embodiment, the perfusion rate is at least about 0.15 V/d to at least about 0.5 V/d. In one embodiment, the perfusion rate is at least about 0.15 V/d to at least about 0.45 V/d. In one embodiment, the perfusion rate is at least about 0.15 V/d to at least about 0.40 V/d. In one embodiment, the perfusion rate is at least about 0.15 V/d to at least about 0.35 V/d. In one embodiment, the perfusion rate is at least about 0.15 V/d to at least about 0.30 V/d. In one embodiment, the perfusion rate is at least about 0.15 V/d to at least about 0.25 V/d. In one embodiment, the perfusion rate is at least about 0.15 V/d to at least about 0.20 V/d.

In one embodiment, the perfusion rate is at least about 0.20 V/d to at least about 0.5 V/d. In one embodiment, the perfusion rate is at least about 0.20 V/d to at least about 0.45 V/d. In one embodiment, the perfusion rate is at least about 0.20 V/d to at least about 0.40 V/d. In one embodiment, the perfusion rate is at least about 0.20 V/d to at least about 0.35 V/d. In one embodiment, the perfusion rate is at least about 0.20 V/d to at least about 0.30 V/d. In one embodiment, the perfusion rate is at least about 0.20 V/d to at least about 0.25 V/d.

In one embodiment, the perfusion rate is at least about 0.3 V/d to at least about 0.5 V/d. In one embodiment, the perfusion rate is at least about 0.3 V/d to at least about 0.45 V/d. In one embodiment, the perfusion rate is at least about 0.3 V/d to at least about 0.40 V/d. In one embodiment, the perfusion rate is maintained at least about 0.30 V/d to at least about 0.35 V/d.

In one embodiment, the perfusion rate is at least about 0.40 V/d to at least about 0.5 V/d. In one embodiment, the perfusion rate is at least about 0.40 V/d to at least about 0.45 V/d.

In one embodiment, the perfusion rate is about 0.05 V/d to about 0.5 V/d. In one embodiment, the perfusion rate is about 0.05 V/d to about 0.45 V/d. In one embodiment, the perfusion rate is about 0.05 V/d to about 0.40 V/d. In one embodiment, the perfusion rate is about 0.05 V/d to about 0.35 V/d. In one embodiment, the perfusion rate is about 0.05 V/d to about 0.30 V/d. In one embodiment, the perfusion rate is about 0.05 V/d to about 0.25 V/d. In one embodiment, the perfusion rate is about 0.05 V/d to about 0.20 V/d. In one embodiment, the perfusion rate is about 0.05 V/d to about 0.15 V/d. In one embodiment, the perfusion rate is about 0.05 V/d to about 0.10 V/d. In one embodiment, the perfusion rate is about 0.1 V/d to about 0.5 V/d. In one embodiment, the perfusion rate is about 0.10 V/d to about 0.45 V/d. In one embodiment, the perfusion rate is about 0.10 V/d to about 0.40 V/d. In one embodiment, the perfusion rate is about 0.10 V/d to about 0.35 V/d. In one embodiment, the perfusion rate is about 0.10 V/d to about 0.30 V/d. In one embodiment, the perfusion rate is about 0.10 V/d to about 0.25 V/d. In one embodiment, the perfusion rate is about 0.10 V/d to about 0.20 V/d. In one embodiment, the perfusion rate is about 0.10 V/d to about 0.15 V/d. In one embodiment, the perfusion rate is about 0.10 V/d to about 0.125 V/d. In one embodiment, the perfusion rate is about 0.15 V/d to about 0.5 V/d. In one embodiment, the perfusion rate is about 0.15 V/d to about 0.45 V/d. In one embodiment, the perfusion rate is about 0.15 V/d to about 0.40 V/d. In one embodiment, the perfusion rate is about 0.15 V/d to about 0.35 V/d. In one embodiment, the perfusion rate is about 0.15 V/d to about 0.30 V/d. In one embodiment, the perfusion rate is about 0.15 V/d to about 0.25 V/d. In one embodiment, the perfusion rate is about 0.15 V/d to about 0.20 V/d. In one embodiment, the perfusion rate is about 0.20 V/d to about 0.5 V/d. In one embodiment, the perfusion rate is about 0.20 V/d to about 0.45 V/d. In one embodiment, the perfusion rate is about 0.20 V/d to about 0.40 V/d. In one embodiment, the perfusion rate is about 0.20 V/d to about 0.35 V/d. In one embodiment, the perfusion rate is about 0.20 V/d to about 0.30 V/d. In one embodiment, the perfusion rate is about 0.20 V/d to about 0.25 V/d. In one embodiment, the perfusion rate is about 0.3 V/d to about 0.5 V/d. In one embodiment, the perfusion rate is about 0.3 V/d to about 0.45 V/d. In one embodiment, the perfusion rate is about 0.3 V/d to about 0.40 V/d. In one embodiment, the perfusion rate is about 0.30 V/d to about 0.35 V/d. In one embodiment, the perfusion rate is about 0.40 V/d to about 0.5 V/d. In one embodiment, the perfusion rate is about 0.40 V/d to about 0.45 V/d. In any of these embodiments, the feed rate and the permeate rate are the same. In any of these embodiments, the culture is perfused at the same rate during the growth phase and the production phase.

In one embodiment, the perfusion rate is at least about 0.10 V/d to 0.40 V/d. In one embodiment, the perfusion rate is at least about 0.10 V/d to 0.25 V/d. In one embodiment, the perfusion rate is at least about 0.13 V/d to 0.39 V/d. In one embodiment, the perfusion rate is at least about 0.13 V/d to 0.22 V/d.

In one embodiment, the perfusion rate is at least about 0.05 V/d, 0.10 V/d, 0.11 V/d, 0.12 V/d, 0.13 V/d, 0.14 V/d, 0.15 V/d, 0.16 V/d, 0.17 V/d, 0.18 V/d, 0.19 V/d, 0.20 V/d, 0.21 V/d, 0.22 V/d, 0.23 V/d, 0.24 V/d, 0.25 V/d, 0.26 V/d, 0.27 V/d, 0.28 V/d, 0.29 V/d, 0.30 V/d, 0.31 V/d, 0.32 V/d, 0.33 V/d, 0.34 V/d, 0.35 V/d, 0.36 V/d, 0.37 V/d, 0.38 V/d, 0.39 V/d, 0.40 V/d, 0.41 V/d, 0.42 V/d, 0.43 V/d, 0.44 V/d, 0.45 V/d, 0.46 V/d, 0.47 V/d, 0.48 V/d, 0.49 V/d, or 0.50 V/d. In one embodiment, the perfusion rate is at least about 0.13 V/d. In one embodiment, the perfusion rate is at least about 0.22 V/d.

In one embodiment, the perfusion rate is about 0.05 V/d, 0.06 V/d, 0.07 V/d, 0.08 V/d, 0.09 V/d, about 0.10 V/d, about 0.11 V/d, about 0.12 V/d, about 0.13 V/d, about 0.14 V/d, about 0.15 V/d, about 0.16 V/d, about 0.17 V/d, about 0.18 V/d, about 0.19 V/d, about 0.20 V/d, about 0.21 V/d, about 0.22 V/d, about 0.23 V/d, about 0.24 V/d, about 0.25 V/d, about 0.26 V/d, about 0.27 V/d, about 0.28 V/d, about 0.29 V/d, about 0.30 V/d, about 0.31 V/d, about 0.32 V/d, about 0.33 V/d, about 0.34 V/d, about 0.35 V/d, about 0.36 V/d, about 0.37 V/d, about 0.38 V/d, about 0.39 V/d, about 0.40 V/d, about 0.41 V/d, about 0.42 V/d, about 0.43 V/d, about 0.44 V/d, about 0.45 V/d, about 0.46 V/d, about 0.47 V/d, about 0.48 V/d, about 0.49 V/d, or about 0.50 V/d. In one embodiment, the perfusion rate is about 0.13 V/d. In one embodiment, the perfusion rate is about 0.22 V/d. In any of these embodiments, the feed rate and the permeate rate are the same. In any of these embodiments, the culture is perfused at the same rate during the growth phase and the production phase.

The growth phase is conducted at a high cell density in a culture volume that is at least 50% to about 75% of the final working volume of the bioreactor, allowing for efficient use of the reduced culture volume, decreasing the amount of feed required and waste generated. It also shortens the growth phase to less than 70% of the culture duration. Introducing a feed up phase transition between the growth phase and the production phase increases the culture volume and decreases the cell mass in the bioreactor, which reduces the amount of feed required and decreases the waste in the permeate flow during the production phase.

When the culture achieves one or more desired target criteria, at least one feed up phase is initiated to increase the culture volume in the bioreactor to a desired final culture volume that is less than or equal to 100% of the final bioreactor working volume. Such target criteria can include one or more time points (e.g., time post inoculation), titer, cell density, packed cell volume, product attribute, process, production and/or a plant schedule.

One of skill in the art can determine a suitable method for increasing the culture volume in a bioreactor. In one embodiment, “differential perfusion” is used to increase the culture volume. During differential perfusion, the feed flow rate and the permeate flow rate are not identical. When the feed flow rate is greater than the permeate rate, a net increase in culture volume occurs.

The feed rate and the permeate rate can be determined such that the desired culture volume is achieved within a desired time frame. In one embodiment, during the feed up phase, one or more feed rates may be used to achieve a desired culture volume in the bioreactor. In one embodiment, the feed rate is increased relative to the permeate rate. In one embodiment, the permeate rate is held constant and the feed rate is increased during the feed up phase. In one embodiment, the permeate rate is decreased relative to the feed rate. In one embodiment, no permeate is collected until the desired culture volume is achieved.

During the feed up phase, the feed and permeate rates are each independently maintained at rates less than or equal to 0.5 culture volumes/day (V/d), with the feed rate being greater than the permeate rate. In one embodiment, the feed rate or the permeate rate is at least about 0.05 V/d to 0.50 V/d. In one embodiment, the feed rate or the permeate rate is at least about 0.05 V/d to at least about 0.45 V/d. In one embodiment, the feed rate or the permeate rate is at least about 0.05 V/d to at least about 0.40 V/d. In one embodiment, the feed rate or the permeate rate is at least about 0.05 V/d to at least about 0.35 V/d. In one embodiment, the feed rate or the permeate rate is at least about 0.05 V/d to at least about 0.30 V/d. In one embodiment, the feed rate or the permeate rate is at least about 0.05 V/d to at least about 0.25 V/d. In one embodiment, the feed rate or the permeate rate is at least about 0.05 V/d to at least about 0.20 V/d. In one embodiment, the feed rate or the permeate rate is at least about 0.05 V/d to at least about 0.15 V/d. In one aspect, the feed rate or the permeate rate is at least about 0.05 V/d to at least about 0.10 V/d.

In one embodiment, the feed rate or the permeate rate is about 0.05 V/d to about 0.50 V/d. In one embodiment, the feed rate or the permeate rate is about 0.05 V/d to about 0.45 V/d. In one embodiment, the feed rate or the permeate rate is about 0.05 V/d to about 0.40 V/d. In one embodiment, the feed rate or the permeate rate is about 0.05 V/d to about 0.35 V/d. In one embodiment, the feed rate or the permeate rate is about 0.05 V/d to about 0.30 V/d. In one embodiment, the feed rate or the permeate rate is about 0.05 V/d to about 0.25 V/d. In one embodiment, the feed rate or the permeate rate is about 0.05 V/d to about 0.20 V/d. In one embodiment, the feed rate or the permeate rate is about 0.05 V/d to about 0.15 V/d. In one aspect, the feed rate or the permeate rate is about 0.05 V/d to about 0.10 V/d.

In one embodiment, the feed rate is at least 0.10 V/d to at least 0.50 V/d. In one embodiment, the feed rate is at least 0.10 V/d to at least 0.45 V/d. In one embodiment, the feed rate is at least 0.10 V/d to at least 0.40 V/d. In one embodiment, the feed rate is at least 0.10 V/d to at least 0.35 V/d. In one embodiment, the feed rate is at least 0.10 V/d to at least 0.30 V/d. In one embodiment, the feed rate is at least 0.10 V/d to at least 0.250 V/d. In one embodiment, the feed rate is at least 0.10 V/d to at least 0.20 V/d. In one embodiment, the feed rate is at least 0.10 V/d to at least 0.15 V/d. In one embodiment, the feed rate is at least 0.33 V/d. In one embodiment, the feed rate is at least 0.22 V/d. In one embodiment, the feed rate is at least 0.10 V/d, 0.11 V/d, 0.12 V/d, 0.13 V/d, 0.14 V/d, 0.15 V/d, 0.16 V/d, 0.17 V/d, 0.18 V/d, 0.19 V/d, 0.20 V/d, 0.21 V/d, 0.22 V/d, 0.23 V/d, 0.24 V/d, 0.25 V/d, 0.26 V/d, 0.27 V/d, 0.28 V/d, 0.29 V/d, 0.30 V/d, 0.31 V/d, 0.32 V/d, 0.33 V/d, 0.34 V/d, 0.35 V/d, 0.36 V/d, 0.37 V/d, 0.38 V/d, 0.39 V/d, 0.40 V/d, 0.45 V/d, or 0.50 V/d.

In one embodiment, the feed rate is about 0.10 V/d to about 0.50 V/d. In one embodiment, the feed rate is about 0.10 V/d to about 0.45 V/d. In one embodiment, the feed rate is about 0.10 V/d to about 0.40 V/d. In one embodiment, the feed rate is about 0.10 V/d to about 0.35 V/d. In one embodiment, the feed rate is about 0.10 V/d to about 0.30 V/d. In one embodiment, the feed rate is about 0.10 V/d to about 0.250 V/d. In one embodiment, the feed rate is about 0.10 V/d to about 0.20 V/d. In one embodiment, the feed rate is about 0.10 V/d to about 0.15 V/d. In one embodiment, the feed rate about 0.33 V/d. In one embodiment, the feed rate is about 0.22 V/d. In one embodiment, the feed rate is about 0.10 V/d, about 0.11 V/d, about 0.12 V/d, about 0.13 V/d, about 0.14 V/d, about 0.15 V/d, about 0.16 V/d, about 0.17 V/d, about 0.18 V/d, about 0.19 V/d, about 0.20 V/d, about 0.21 V/d, about 0.22 V/d, about 0.23 V/d, about 0.24 V/d, about 0.25 V/d, about 0.26 V/d, about 0.27 V/d, about 0.28 V/d, about 0.29 V/d, about 0.30 V/d, about 0.31 V/d, about 0.32 V/d, about 0.33 V/d, about 0.34 V/d, about 0.35 V/d, about 0.36 V/d, about 0.37 V/d, about 0.38 V/d, about 0.39 V/d, about 0.40 V/d, about 0.45 V/d, or about 0.50 V/d.

In one embodiment, the permeate rate is 0 V/d to at least 0.50 V/d. In one embodiment, the permeate rate is at least about 0.45 V/d. In one embodiment, the permeate rate is at least about 0.40 V/d. In one embodiment, the permeate rate is at least about 0.10 V/d to at least about 0.35 V/d. In one embodiment, the permeate rate is at least about 0.10 V/d to about 0.30 V/d. In one aspect, the permeate rate is at least about 0.10 V/d to about 0.25 V/d. In one embodiment, the permeate rate is at least about 0.10 V/d to about 0.20 V/d. In one embodiment, the permeate rate is at least about 0.10 V/d to about 0.15 V/d. In one embodiment, the permeate rate is at least about 0.13 V/d. In one embodiment, the permeate rate is at least 0 V/d, 0.10 V/d, 0.11 V/d, 0.12 V/d, 0.13 V/d, 0.14 V/d, 0.15 V/d, 0.16 V/d, 0.17 V/d, 0.18 V/d, 0.19 V/d, 0.20 V/d, 0.21 V/d, 0.22 V/d, 0.23 V/d, 0.24 V/d, 0.25 V/d, 0.26 V/d, 0.27 V/d, 0.28 V/d, 0.29 V/d, or 0.30 V/d.

In one embodiment, the permeate rate is 0 V/d to about 0.50 V/d. In one embodiment, the permeate rate is 0 V/d to about 0.40 V/d. In one embodiment, the permeate rate is about 0.10 V/d to about 0.35 V/d. In one embodiment, the permeate rate is about 0.10 V/d to about 0.30 V/d. In one embodiment, the permeate rate is about 0.10 V/d to about 0.25 V/d. In one embodiment, the permeate rate is about 0.10 V/d to about 0.20 V/d. In one embodiment, the permeate rate is about 0.10 V/d to about 0.15 V/d. In one embodiment, the permeate rate is about 0 V/d, about 0.10 V/d, about 0.11 V/d, about 0.12 V/d, about 0.13 V/d, about 0.14 V/d, about 0.15 V/d, about 0.16 V/d, about 0.17 V/d, about 0.18 V/d, about 0.19 V/d, about 0.20 V/d, about 0.21 V/d, about 0.22 V/d, about 0.23 V/d, about 0.24 V/d, about 0.25 V/d, about 0.26 V/d, about 0.27 V/d, about 0.28 V/d, about 0.29 V/d, or about 0.30 V/d. In one embodiment, the permeate rate is about 0.45 V/d. In one embodiment, the permeate rate is about 0.40 V/d. In one embodiment, the permeate rate is about 0.13 V/d.

In one embodiment, the feed rate is at least 0.30 V/d and the permeate rate is less than 0.20 V/d. In one embodiment, the feed rate is less than 0.40 V/d and the permeate rate is less than 0.20 V/d. In one embodiment, the feed rate is between 0.3 V/d and 0.4 V/d and the permeate rate is between 0.10 V/d and 0.15 V/d. In one embodiment, the feed rate is about 0.3 V/d to about 0.4 V/d and the permeate rate is about 0.1 V/d to about 0.2 V/d.

In one embodiment, the feed rate during the feed up phase is selected to attain or achieve one or more desired target criteria, such as a culture volume, final culture volume, bioreactor working volume, final bioreactor working volume, time point, titer, cell density, packed cell volume, product attribute, process, production and/or a plant schedule.

In one embodiment, one or more feed rates are selected such that the feed up phase is maintained for at least 6 hours. In one embodiment, the feed up phase is maintained for at least 60 hours. In one embodiment, the feed up period is maintained for at least about 16 hours to at least about 40 hours. In one embodiment, the feed up phase is maintained for 30 hours ±24 hours. In one embodiment, the feed up phase is maintained for 30 hours ±8 hours. In one embodiment, the feed up phase is maintained for at least 6, 10, 12, 16, 20, 21, 22, 24, 25, 26, 27, 28, 29, 30, 31, 32, 33, 34, 35, 36, 37, 38, 39, 40, 48, 50, 54, or 60 hours.

In one embodiment, the feed up phase is maintained for about 6 hours to about 60 hours (such as, e.g., about 12 hours to about 48 hours, about 16 hours to about 40 hours, about 6 hours to about 54 hours). In one embodiment, the feed up phase is maintained for about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 48, about 50, about 54, or about 60 hours.

When the feed up phase is complete, the feed rate and the permeate rate can revert to an identical flow rate of less than or equal to 0.5 V/d until harvest. In one embodiment, the feed rate reverts to the same rate as the permeate rate. In one embodiment, the feed and permeate rate revert to the rate prior to the feed up phase (i.e., the feed and permeate rates during the preceding growth phase, which can be the same).

In one embodiment, the feed rate and the permeate rate are at least about 0.05 V/d. In one embodiment, the feed rate and the permeate rate are at least about 0.05 V/d to less than 0.5 V/d. In one embodiment, the feed rate and the permeate rate are at least about 0.05 V/d to at least about 0.45 V/d. In one embodiment, the feed rate and the permeate rate are at least about 0.05 V/d to at least about 0.40 V/d. In one embodiment, the feed rate and the permeate rate are at least about 0.05 V/d to at least about 0.35 V/d. In one embodiment, the feed rate and the permeate rate are at least about 0.05 V/d to at least about 0.30 V/d. In one embodiment, the feed rate and the permeate rate are at least about 0.05 V/d to at least about 0.25 V/d. In one embodiment, the feed rate and the permeate rate are at least about 0.05 V/d to at least about 0.20 V/d. In one embodiment, the feed rate and the permeate rate are at least about 0.05 V/d to at least about 0.15 V/d. In one embodiment, the feed rate and the permeate rate are at least about 0.05 V/d to at least about 0.10 V/d. In one embodiment, the feed rate and the permeate rate are at least about 0.1 V/d to 0.5 V/d. In one embodiment, the feed rate and the permeate rate are at least about 0.10 V/d to at least about 0.45 V/d. In one embodiment, the feed rate and the permeate rate are at least about 0.10 V/d to at least about 0.40 V/d. In one embodiment, the feed rate and the permeate rate are at least about 0.10 V/d to at least about 0.35 V/d. In one embodiment, the feed rate and the permeate rate are at least about 0.10 V/d to at least about 0.30 V/d. In one embodiment, the feed rate and the permeate rate are at least about 0.10 V/d to at least about 0.25 V/d. In one embodiment, the feed rate and the permeate rate are at least about 0.10 V/d to at least about 0.20 V/d. In one embodiment, the feed rate and the permeate rate are at least about 0.10 V/d to at least about 0.15 V/d. In any of these embodiments, the feed rate and the permeate rate can be the same.

In one embodiment, the feed rate and the permeate rate are increased to a rate greater than 0.20 V/d 24 hours following the end of the feed up phase. In one embodiment, the feed rate and the permeate rate are 0.05 V/d. In one embodiment, the feed rate and the permeate rate are 0.10 V/d. In one embodiment, the feed rate and the permeate rate are 0.15 V/d. In one embodiment, the feed rate and the permeate rate are 0.20 V/d. In one embodiment, the feed rate and the permeate rate are 0.25 V/d. In one embodiment, the feed rate and the permeate rate are 0.30 V/d. In one embodiment, the feed rate and the permeate rate are 0.35 V/d. In one embodiment, the feed rate and the permeate rate are 0.40 V/d. In one embodiment, the feed rate and the permeate rate are 0.45 V/d. In one embodiment, the feed rate and the permeate rate are 0.50 V/d.

In one embodiment, 24 hours following the end of the feed up phase, the feed rate and the permeate rate are the same and are a rate in the range of about 0.05 V/d to about 0.5 V/d. In one embodiment, 24 hours following the end of the feed up phase, the feed rate and the permeate rate are the same and are a rate in the range of about 0.05 V/d to about 0.45 V/d. In one embodiment, 24 hours following the end of the feed up phase, the feed rate and the permeate rate are the same and are a rate in the range of about 0.05 V/d to about 0.40 V/d. In one embodiment, 24 hours following the end of the feed up phase, the feed rate and the permeate rate are the same and are a rate in the range of about 0.05 V/d to about 0.35 V/d. In one embodiment, 24 hours following the end of the feed up phase, the feed rate and the permeate rate are the same and are a rate in the range of about 0.05 V/d to about 0.30 V/d. In one embodiment, 24 hours following the end of the feed up phase, the feed rate and the permeate rate are the same and are a rate in the range of about 0.05 V/d to about 0.25 V/d. In one embodiment, 24 hours following the end of the feed up phase, the feed rate and the permeate rate are the same and are a rate in the range of about 0.05 V/d to about 0.20 V/d. In one embodiment, 24 hours following the end of the feed up phase, the feed rate and the permeate rate are the same and are a rate in the range of about 0.05 V/d to about 0.15 V/d. In one embodiment, the feed rate and the permeate rate are the same and are a rate in the range of about 0.05 V/d to about 0.10 V/d. In one embodiment, 24 hours following the end of the feed up phase, the feed rate and the permeate rate are the same and are a rate in the range of about 0.1 V/d to about 0.5 V/d. In one embodiment, 24 hours following the end of the feed up phase, the feed rate and the permeate rate are the same and are a rate in the range of about 0.10 V/d to about 0.45 V/d. In one embodiment, 24 hours following the end of the feed up phase, the feed rate and the permeate rate are the same and are a rate in the range of about 0.10 V/d to about 0.40 V/d. In one embodiment, 24 hours following the end of the feed up phase, the feed rate and the permeate rate are the same and are a rate in the range of about 0.10 V/d to about 0.35 V/d. In one embodiment, 24 hours following the end of the feed up phase, the feed rate and the permeate rate are the same and are a rate in the range of about 0.10 V/d to about 0.30 V/d. In one embodiment, 24 hours following the end of the feed up phase, the feed rate and the permeate rate are the same and are a rate in the range of about 0.10 V/d to about 0.25 V/d. In one embodiment, 24 hours following the end of the feed up phase, the feed rate and the permeate rate are the same and are a rate in the range of about 0.10 V/d to about 0.20 V/d. In one embodiment, 24 hours following the end of the feed up phase, the feed rate and the permeate rate are the same and are a rate in the range of about 0.10 V/d to about 0.15 V/d.

In one embodiment, the feed rate and the permeate rate are the same and are about 0.05 V/d, about 0.10 V/d, about 0.15 V/d, about 0.20 V/d, about 0.25 V/d, about 0.30 V/d, about 0.35 V/d, about 0.4 V/d, about 0.45 V/d, or about 0.5 V/d 24 hours following the end of the feed up phase. In one embodiment, the feed rate and the permeate rate are the same and are about 0.25 V/d, about 0.30 V/d, about 0.35 V/d, about 0.4 V/d, about 0.45 V/d, or about 0.5 V/d 24 hours following the end of the feed up phase. In one embodiment, the feed rate and the permeate rate are the same and are about 0.05 V/d, about 0.10 V/d, about 0.15 V/d, or about 0.20 V/d 24 hours following the end of the feed up phase.

In one embodiment, the feed up phase is initiated when the culture achieves one or more desired target criteria, including a desired culture volume, final culture volume, bioreactor working volume, final bioreactor working volume, time point, titer, cell density, packed cell volume, product attribute, process, production and/or a plant schedule.

In one embodiment, the feed up phase is initiated when the culture reaches a desired cell density. In one embodiment, the feed up phase is initiated when the cell density is at least 100×10⁵ cells/mL. In one embodiment, the cell density is up to 350×10⁵ cells/mL. In one embodiment, the cell density is at least about 100×10⁵ to about 350×10⁵ cells/mL. In one embodiment, the cell density is at least about 100×10⁵ to 300×10⁵ cells/mL. In one embodiment, the cell density is at least about 100×10⁵ to 250×10⁵ cells/mL. In one embodiment, the cell density is at least about 100×10⁵ to 200×10⁵ cells/mL. In one embodiment, the cell density is at least about 100×10⁵ to 150×10⁵ cells/mL. In one embodiment, the cell density is at least about 100×10⁵, 125×10⁵, 150×10⁵, 175×10⁵, 200×10⁵, 225×10⁵, 250×10⁵, 275×10⁵, 300×10⁵, 325×10⁵, or 350×10⁵ cells/mL. In one embodiment, the cell density is at least about 100×10⁵ cells/mL. In one embodiment, the cell density is at least about 150×10⁵ cells/mL. In one embodiment, the cell density is at least about 200×10⁵ cells/mL. In one embodiment, the cell density is at least about 250×10⁵ cells/mL. In one embodiment, the cell density is at least about 300×10⁵ cells/mL. In one embodiment, the cell density is about 350×10⁵ cells/mL.

In one embodiment, the feed up phase is initiated when the culture reaches a cell density in the range of about 100×10⁵ cells/mL to about 350×10⁵ cells/mL (e.g., about 100×10⁵, about 125×10⁵, about 150×10⁵, about 175×10⁵, about 200×10⁵, about 225×10⁵, about 250×10⁵, about 275×10⁵, about 300×10⁵, about 325×10⁵, or about 350×10⁵ cells/mL).

In one embodiment, the feed up phase is initiated at least about 24 hours post-inoculation. In one embodiment, the feed up phase is initiated up to 96 hours post-inoculation. In one embodiment, the feed up phase is initiated about 24 hours to about 96 hours post-inoculation. In one embodiment, the feed up phase is initiated about 24 hours to about 72 hours post-inoculation. In one embodiment, the feed up phase is initiated about 24 hours to about 48 hours post-inoculation. In one embodiment, the feed up phase is initiated about 48 hours to about 96 hours post-inoculation. In one embodiment, the feed up phase is initiated about 48 hours to about 72 hours post-inoculation. In one embodiment, the feed up phase is initiated about 72 hours to about 96 hours post-inoculation. In one embodiment, the feed up phase is initiated about 24, 48, 72, or 96 hours post-inoculation. In one embodiment, the feed up phase is initiated about 24 hours post-inoculation. In one embodiment, the feed up phase is initiated about 48 hours post-inoculation. In one embodiment, the feed up phase is initiated about 72 hours post-inoculation. In one embodiment, the feed up phase is initiated about 96 hours post-inoculation.

The feed up phase is maintained until one or more desired target criteria is met. Such target criteria include, but are not limited to, a desired culture volume, a final culture volume, a desired bioreactor working volume, a final bioreactor working volume, time point, titer, cell density, packed cell volume, product attribute, process, production and/or a plant schedule.

In one embodiment, the feed up phase is maintained for a desired length of time. In one embodiment, the desired time is based on a process, production and/or plant schedule. In one embodiment, the feed up phase is maintained for such a time as to allow for at least one population doubling of the culture. In one embodiment, the feed up phase is maintained for a sufficient time such that the culture is not shocked due to the rapid increase in media concentrates.

In one embodiment, the feed up phase is maintained until a desired culture volume is achieved. In one embodiment, the desired culture volume is equal to the final working volume of the bioreactor. In one embodiment, the feed up phase is maintained until the culture volume is greater than 70% of the final working volume of the bioreactor. In one embodiment, the feed up phase is maintained until the culture volume is greater than 75% of the final working volume of the bioreactor. In one embodiment, the feed up is maintained until the culture volume is greater than 75% to less than or equal to 100% of the final working volume of the bioreactor. In one embodiment, the feed up is maintained until the culture volume is about 80% final working volume of the bioreactor to less than or equal to 100% final working volume of the bioreactor. In one embodiment, the feed up is maintained until the culture volume is about 85% final working volume of the bioreactor to less than or equal to 100% final working volume of the bioreactor. In one embodiment, the feed up is maintained until the culture volume is about 90% final working volume of the bioreactor to less than or equal to 100% final working volume of the bioreactor. In one embodiment, the feed up is maintained until the culture volume is about 90% final working volume of the bioreactor. In one embodiment, the feed up is maintained until the culture volume is about 910% final working volume of the bioreactor. In one embodiment, the feed up is maintained until the culture volume is about 92% final working volume of the bioreactor. In one embodiment, the feed up is maintained until the culture volume is about 93% final working volume of the bioreactor. In one embodiment, the feed up is maintained until the culture volume is about 94% final working volume of the bioreactor. In one embodiment, the feed up is maintained until the culture volume is about 95% final working volume of the bioreactor. In one embodiment, the feed up is maintained until the culture volume is about 96% final working volume of the bioreactor. In one embodiment, the feed up is maintained until the culture volume is about 97% final working volume of the bioreactor. In one embodiment, the feed up is maintained until the culture volume is about 98% final working volume of the bioreactor. In one embodiment, the feed up is maintained until the culture volume is about 99% final working volume of the bioreactor. In one embodiment, the feed up is maintained until the culture volume is 100% final working volume of the bioreactor.

The feed up phase may be maintained until a desired cell density in the increased culture volume is achieved. In one embodiment, the feed up phase is maintained until a desired cell density is achieved at a desired cell culture volume. In one embodiment, the feed up phase is maintained until the cell density is at least about 200×10⁵ cells/mL. In one embodiment, the feed up phase is maintained until the cell density is about 600×10⁵ cells/mL. In one embodiment, the feed up phase is maintained until the cell density is at least about 200×10⁵ to about 600×10⁵ cells/mL. In one embodiment, the feed up phase is maintained until the cell density is at least about 200×10⁵ to at least about 550×10⁵ cells/mL. In one embodiment, the feed up phase is maintained until the cell density is at least about 200×10⁵ to at least about 500×10⁵ cells/mL. In one embodiment, the feed up phase is maintained until the cell density is at least about 200×10⁵ to at least about 450×10⁵ cells/mL. In one embodiment, the feed up phase is maintained until the cell density is at least about 200×10⁵ to at least about 400×10⁵ cells/mL. In one embodiment, the feed up phase is maintained until the cell density is at least about 200×10⁵ to at least about 350×10⁵ cells/mL. In one embodiment, the feed up phase is maintained until the cell density is at least about 200×10⁵ to at least about 300×10⁵ cells/mL. In one embodiment, the feed up phase is maintained until the cell density is at least about 200×10⁵ to at least about 250×10⁵ cells/mL. In one embodiment, the feed up phase is maintained until the cell density is at least about 300×10⁵ to at least 600×10⁵ cells/mL. In one embodiment, the feed up phase is maintained until the cell density is at least about 400×10⁵ to at least 600×10⁵ cells/mL. In one embodiment, the feed up phase is maintained until the cell density is at least about 500×10⁵ to at least 600×10⁵ cells/mL. In one embodiment, the feed up phase is maintained until the cell density is at least about 200×10⁵, 225×10⁵, 250×10⁵, 275×10⁵, 300×10⁵, 325×10⁵, 350×10⁵, 375×10⁵, 400×10⁵, 425×10⁵, 450×10⁵, 475×10⁵, 500×10⁵, 525×10⁵, 550×10⁵, 575×10⁵, or 600×10⁵ cells/mL. In one embodiment, the feed up phase is maintained until the cell density is at least about 300×10⁵ cells/mL. In one embodiment, the feed up phase is maintained until the cell density is at least about 400×10⁵ cells/mL. In one embodiment, the feed up phase is maintained until the cell density is at least about 500×10⁵ cells/mL.

In one embodiment, the feed up phase is maintained until the cell density is about 200×10⁵ cells/mL to about 600×10⁵ cells/mL (e.g., about 200×10⁵, about 225×10⁵, about 250×10⁵, about 275×10⁵, about 300×10⁵, about 325×10⁵, about 350×10⁵, about 375×10⁵, about 400×10⁵, about 425×10⁵, about 450×10⁵, about 475×10⁵, about 500×10⁵, about 525×10⁵, about 550×10⁵, about 575×10⁵, or about 600×10⁵ cells/mL).

In one embodiment, the growth phase continues during and after the feed up phase. In one embodiment, the growth phase continues until a desired cell density is achieved following the feed up phase. In one embodiment, the growth phase continues following a temperature shift.

The production phase marks the start of the stationary phase in which cell growth typically levels off and product titer increases. In one embodiment, the production phase begins when the feed up phase has ended. In one embodiment, the production phase begins when a desired cell culture volume, final cell culture volume, bioreactor working volume, final bioreactor working volume, time point, titer, cell density, packed cell volume, product attribute, process, production and/or a plant schedule, has been achieved.

In one embodiment, the production phase begins when a desired cell density is achieved. In one embodiment, the desired cell density is at least 200×10⁵ cells/mL. In one embodiment, the desired cell density is at least 250×10⁵ cells/mL. In one embodiment, the desired cell density is at least 300×10⁵ cells/mL. In one embodiment, the desired cell density is at least 350×10⁵ cells/mL. In one embodiment, the desired cell density is at least 400×10⁵ cells/m. In one embodiment, the desired cell density is at least 500×10⁵ cells/mL. In one embodiment, the desired cell density is at least 550×10⁵ cells/mL. In one embodiment, the desired cell density is at least 600×10⁵ cells/mL.

In one embodiment, the production phase begins once a desired cell density at a desired culture volume or a desired working volume is achieved. In one embodiment, the production phase begins once a desired cell density is achieved at a desired culture volume. In one embodiment, the production phase begins once a desired cell density is achieved at a desired final culture volume. In one embodiment, the production phase begins once a desired cell density is achieved at a desired working volume of the bioreactor. In one embodiment, the production phase begins once a desired cell density is achieved at the desired final working volume of the bioreactor. In one embodiment, the production phase begins when the cell density is at least 200×10⁵ cells/mL in the final culture volume. In one embodiment, the production phase begins when the cell density is at least 200×10⁵ cells/mL in the final working volume of the bioreactor.

In one embodiment, the production phase begins when the cell density is at least 2 times the inoculation cell density. In one embodiment, the production phase begins when the cell density is less than or equal to 3 times the inoculation cell density.

In one embodiment, the production phase begins when the culture volume is greater than 75% to less than or equal to 100% of the final working volume of the bioreactor.

To increase production of a desired protein by the mammalian cells, cell growth can be limited or arrested. Such methods include, but are not limited to, temperature shifts, pH shifts, use of chemical inducers of protein production and cell cycle inhibitors, nutrient limitation, or starvation, either alone or in combination. For example, lower culture temperature(s) may be used to decrease cell growth and to promote protein production and culture longevity. Use of temperature shifts from optimal growth phase temperatures to optimal production phase temperatures are often employed in cell culture strategies. To optimize mammalian cell growth, the culture temperature is typically maintained at physiological temperature, 35° C. to 37° C., during the growth phase. The temperature during the production phase may be from about 28° C. to about 35° C. Temperature modulation can be used throughout the duration of the culture to achieve desired growth and production objectives. Combinations of temperature shifts may also be used to go from a first growth phase to a first production phase, to second growth phase, followed by a second production phase, and so on.

In one embodiment, the temperature is shifted mid-culture from an optimal temperature for the growth phase to an optimal temperature for the production phase. In one embodiment, the temperature shift occurs at the end of the growth phase. In one embodiment, the temperature shift occurs at the end of the feed up phase. In one embodiment, the temperature shift occurs upon reaching a desired cell density. In one embodiment, the desired cell density is at least 200×10⁵ cells/mL. In one embodiment, the temperature shift occurs when the culture volume reaches a desired final bioreactor working volume. In one embodiment, the temperature shift occurs when the culture volume is greater than 75% of the final working volume of the bioreactor.

In one embodiment of the present disclosure, the temperature during the growth phase is from about 35° C. to about 38° C. In one embodiment, the temperature is from about 34° C. to about 36° C. In one embodiment, the temperature is at least 35° C., 36° C., or 37° C.±0.5° C. In one embodiment, the temperature is 38° C., 37° C., 36.5° C., 36° C., 35.5° C., 35° C., or 34.5° C.

In one embodiment of the present disclosure, the temperature during the production phase is from about 32° C. to about 35° C. In one embodiment, the temperature is from about 30° C. to about 35° C. In one embodiment, the temperature is from about 32° C. to about 34° C. In one embodiment, the temperature is at least 30° C., 31° C., 32° C., 33° C., or 34° C.±0.5° C. In one embodiment, the temperature is 32° C., 32.5° C., 33° C., or 33.5° C.

In one embodiment, at least one temperature shift following the feed up phase is beneficial to maintaining the cell density during the production phase.

In one embodiment, the production phase begins when the culture temperature is decreased.

In one embodiment, a pH shift may be used alone or in combination with a temperature shift.

Another method to maintain cells at a desired physiological state is to induce cell growth-arrest by exposure of the cell culture to low L-asparagine conditions and/or asparagine starvation (see, e.g., WIPO Publication No. WO 2013/006479). Cell growth-arrest may be achieved and maintained through a culture media that contains a limiting concentration of L-asparagine and by maintaining a low concentration of L-asparagine in the cell culture. Maintaining the concentration of L-asparagine at 5 mM or less can be used to induce and maintain cells in a growth-arrested state whereby productivity is increased.

In addition, chemical inducers of protein production, such as caffeine, butyrate, and hexamethylene bisacetamide (HMBA), may be added before, at the same time as, and/or after a temperature shift, or in place of a temperature shift. If inducers are added after a temperature shift, they can be added from one hour to five days after the temperature shift, optionally from one to two days after the temperature shift. Cell cycle inhibitors, compounds known or suspected to regulate cell cycle progression and the associated processes of transcription, DNA repair, differentiation, senescence and apoptosis related to this, are also useful to induce cell growth-arrest. Cell cycle inhibitors that interact with the cycle machinery, such as, e.g., cyclin-dependent kinases (CDKs), are also useful, as are those molecules that interact with proteins from other pathways, such as AKT, mTOR, and other pathways that affect, directly or indirectly, the cell cycle.

Shifts in pH may also be used alone or in combination with temperature and/or chemical inducers.

The production phase continues until one or more desired target criteria, including, e.g., a culture volume, final culture volume, bioreactor working volume, final bioreactor working volume, time point, titer, cell density, packed cell volume, product attribute, process, production and/or a plant schedule, are met.

In one embodiment, the production phase is 50 days or more. In one embodiment, the production phase is less than or equal to 50 days. In one embodiment, the production phase is less than or equal to 40 days. In one embodiment, the production phase is less than or equal to 45 days. In one embodiment, the production phase is less than or equal to 40 days. In one embodiment, the production phase is less than or equal to 35 days. In one embodiment, the production phase is less than or equal to 30 days. In one embodiment, the production phase is less than or equal to 35 days. In one embodiment, the production phase is less than or equal to 30 days. In one embodiment, the production phase is less than or equal to 25 days. In one embodiment, the production phase is less than or equal to 20 days. In one embodiment, the production phase is less than or equal to 19 days. In one embodiment, the production phase is less than or equal to 18 days. In one embodiment, the production phase is less than or equal to 17 days. In one embodiment, the production phase is less than or equal to 16 days. In one embodiment, the production phase is less than or equal to 15 days. In one embodiment, the production phase is less than or equal to 14 days. In one embodiment, the production phase is less than or equal to 13 days. In one embodiment, the production phase is less than or equal to 12 days. In one embodiment, the production phase is less than or equal to 11 days. In one embodiment, the production phase is less than or equal to 10 days. In one embodiment, the production phase is less than or equal to 9 days. In one embodiment, the production phase is less than or equal to 8 days. In one embodiment, the production phase is less than or equal to 7 days. In one embodiment, the production phase is less than or equal to 6 days. In one embodiment, the production phase is less than or equal to 5 days. In one embodiment, the production phase is less than or equal to 4 days.

In one embodiment, the production phase is about 4 days to about 20 days (e.g., about 5 days to about 20 days, about 6 days to about 20 days, about 7 days to about 20 days, about 8 days to about 20 days, about 9 days to about 20 days, about 10 days to about 20 days, about 11 days to about 20 days, about 12 days to about 20 days, about 13 days to about 20 days, about 14 days to about 20 days, about 15 days to about 20 days, about 16 days to about 20 days, about 17 days to about 20 days, about 18 days to about 20 days, about 19 days to about 20 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days).

In one embodiment, the production phase is at least 10-20 days. In one embodiment, the production phase is at least 11-20 days. In one embodiment, the production phase is at least 12-20 days. In one embodiment, the production phase is at least 13-20 days. In one embodiment, the production phase is at least 14-20 days. In one embodiment, the production phase is at least 15-20 days. In one embodiment, the production phase is at least 16-20 days. In one embodiment, the production phase is at least 17-20 days. In one embodiment, the production phase is at least 18-20 days. In one embodiment, the production phase is at least 19-20 days.

In one embodiment, the culture duration, from initiation to harvest, is at least 10 days. In one embodiment, the culture duration is at least 20 days. In one embodiment, the culture duration is at least 30 days. In one embodiment, the culture duration is at least 40 days. In one embodiment, the culture duration is at least 50 days. In one embodiment, the culture duration is at least 60 days. In one embodiment, the culture duration is 10 to 20 days. In one embodiment, the culture duration is 11 to 20 days. In one embodiment, the culture duration is 12 to 20 days. In one embodiment, the culture duration is 13 to 20 days. In one embodiment, the culture duration is 14 to 20 days. In one embodiment, the culture duration is 15 to 20 days. In one embodiment, the culture duration is 16 to 20 days. In one embodiment, the culture duration is 17 to 20 days. In one embodiment, the culture duration is 18 to 20 days. In one embodiment, the culture duration is 19 to 20 days.

Following the production phase, the culture is harvested. Alternatively, or in addition, the contents of the bioreactor may be partially harvested one or more times during the production phase.

Over the duration of the culture, the cell culture is sampled as needed to monitor the culture and operation conditions. One or more conditions, attributes, characterizations, etc., of the culture may be monitored, such as, e.g., cell count, viability, viable cell density, packed cell volume, bioreactor volume, pH, pCO₂, dissolved oxygen (DO), glucose, lactate, ammonia, osmolality, titer, amino acids, and product quality.

The lean perfusion method described herein makes use of one or more separation systems, which are connected to the bioreactor and remove spent media in the permeate flow. In one embodiment, recombinant protein is retained in the bioreactor by the separation system and bulk harvested. In one embodiment, some recombinant protein may be removed from the bioreactor by the separation system during the growth and/or production phases, prior to bulk harvest. In one embodiment, the recombinant protein may be harvested using the separation system.

Separation systems comprise at least one pumping mechanism and at least one filter and/or at least one membrane module. Such separation systems make use of filters including membrane filters, such as, e.g., hollow fiber filters. The pumping mechanism draws spent culture fluid from the bioreactor into the filter or membrane module. The filter or membrane module is used to selectively retain or return certain components (e.g., cells, recombinant protein) in the culture fluid to the bioreactor in the retentate, and remove spent media containing components such as accumulated waste, byproducts, impurities, process- and product-related impurities, and the like in the permeate.

The filter or membrane module in the separation system operates by separating components in the spent culture fluid on the basis of molecular weight of the culture component relative to the pore of the filter or membrane. Tangential Flow Filtration (TFF), also known as crossflow filtration, may be used in separation systems. A feed stream of spent culture fluid passes parallel to the filter or membrane face by means of a pumping mechanism. Those components of the culture that can pass through the membrane exit the separation system in the permeate flow, while the remainder of the culture fluid is recirculated back to the bioreactor in the retentate flow. Tangential flow separation systems can be unidirectional recirculating tangential flow systems (RTF) or alternating tangential flow (ATF) filtration systems.

Hollow fiber filters are commonly used in retention systems. When the spent culture fluid is introduced to the filter, the hollow fiber material may retain certain culture components (including, e.g., cells and the desired protein) on the lumen side (inside) and may allow certain components to pass through the filter in the permeate flow, based on the pore size or molecular weight cutoff of the hollow fiber material. The material that is retained on the lumen side of the filter in the retentate flow is returned to the bioreactor. The material that leaves the separation system in the permeate flow is processed as waste. The separation system can be used to concentrate a desired recombinant protein for harvest. The pore size or molecular weight cut-off (MWCO) may be chosen such that it retains a desired protein in the retentate and returns it back to the bioreactor.

Hollow fibers, in various aspects, have inner diameters of about 0.5 mm to about 1 mm, and may be of any suitable length (such as, e.g., about 30 cm to about 110 cm). Ultrafiltration hollow fibers typically have a pore size range of 0.01 m to 0.1 m or a molecular weight cut off (MWCO) of 300 kDa or less and can be used to retain the desired protein in the retentate and return it back to the bioreactor. In one embodiment, the MWCO is at least 5 kDa to at least 300 kDa. In one embodiment, the MWCO is 5 kDa to at least 100 kDa. In one embodiment, the MWCO is 10 kDa to at least 30 kDa. In one embodiment, the MWCO is at least 5 kDa, 10 kDa, 30 kDa, 50 kDa, 100 kDa, or 300 kDa. In one embodiment, the MWCO is 30 kDa. In one embodiment, the MWCO is about 5 kDa to about 300 kDa (e.g., about 10 kDa to about 300 kDa, about 10 kDa to about 30 kDa, about 5 kDa, about 10 kDa, about 30 kDa, about 50 kDa, about 100 kDa, about 300 kDa). Such filters are available commercially, such as, e.g., ATF6 30 kDa filters and ATF10 30 kDa filters (Refine Technologies, Hanover, NJ), Xampler (Cytiva, Marlborough, MA), Midikros (Spectrum Laboratories, Inc, Dominguez, CA.), XCell ATF®, Repligen, Waltham, MA).

One or more separation systems and/or filters can be used at a time. In one embodiment, two or more separation systems or filters are operated in parallel. In one embodiment, two or more separation systems or filters are operated in series.

Cell culture fluid may be drawn out of the bioreactor and into the filter module by a pumping system, which passes the cell culture through or along a filter and returns the retentate back to the bioreactor. Examples of cell pumping systems include, but are not limited to, peristaltic pumps, double diaphragm pumps, low shear pumps (Levitronix™ pumps, Zurich, Switzerland), reverse tangential flow, and alternating tangential flow systems (ATF™, Repligen, Waltham, MA). In one embodiment, the permeate may be drawn from the filters using peristaltic pumps.

“Cell” or “cells,” as used herein, include any prokaryotic or eukaryotic cell. Cells can be derived ex vivo, in vitro, or in vivo, either separately or as part of a higher structure such as a tissue or organ. Cells are typically derived from a lineage arising from a primary culture that can be maintained in culture for an unlimited time. The selection of an appropriate cell will depend upon various factors, such as, e.g., desired expression levels, protein modifications that are desirable or necessary for activity (such as glycosylation or phosphorylation), and ease of folding into a biologically active molecule.

Preferably cells are eukaryotic cells, such as mammalian cells (such as, e.g., CHO cells). Any mammalian cell suitable for recombinant protein expression is appropriate for use in the context of the disclosure. Suitable mammalian cells include, but are not limited to, Chinese hamster ovary (CHO) cells, human embryonic kidney (HEK) cells, murine myeloma (NS0, Sp2/0) cells, baby hamster kidney (BHK) cells, human embryonic kidney (293) cells, fibrosarcoma (HT-1080) cells, human embryonic retinal (PER.C6) cells, hybrid kidney and B cells (HKB-11), CEVEC's amniocyte production (CAP) cells, human liver (HuH-7) cell, and any other cells that are used or suitable for use in clinical and/or commercial manufacturing. CHO cell lines are widely used to produce complex recombinant proteins. The dihydrofolate reductase (DHFR)-deficient mutant cell lines (Urlaub et al. (1980), Proc Natl Acad Sci USA 77: 4216-4220), DXB 11 and DG-44, are desirable CHO host cell lines because the efficient DHFR selectable and amplifiable gene expression system allows high level recombinant protein expression in these cells (Kaufman R. J. (1990), Meth Enzymol 185:537-566). The glutamine synthetase (GS)-knockout CHOK1SV cell lines, making use of glutamine synthetase (GS)-based methionine sulfoximine (MSX) selection, are also widely used. Also included as cells of the present disclosure are CHOK1 cells (ATCC CCL61).

In a preferred embodiment, the cells are genetically engineered to express a protein of commercial or scientific interest. Methods and materials for genetically engineering cells to express desired proteins are well known to those of skill in the art. As used herein, the term “cell culturing medium” (also referred to as “media,” “culture medium,” “cell culture media,” “tissue culture media,” and the like) refers to any nutrient solution used for growing cells, such as, e.g., mammalian cells. Cell culturing medium generally provides one or more of the following components: an energy source (e.g., in the form of a carbohydrate, such as, e.g., glucose); one or more essential amino acids (e.g., all essential amino acids; the twenty basic amino acids plus cysteine); vitamins and/or other organic compounds typically required at low concentrations; lipids or free fatty acids; and trace elements, such as, e.g., inorganic compounds or naturally occurring elements that are typically required at very low concentrations, such as, e.g., concentrations in the micromolar range. As used herein, cell culturing medium encompasses nutrient solutions that are typically employed in and/or are known for use with any cell culture process, including, but not limited to, batch, extended batch, fed-batch and/or perfusion or continuous culturing of cells.

Various media formulations can be used during the course of a cell culture, for example, to initiate the culture, to facilitate the transition from one stage (e.g., the growth stage or phase) to another (e.g., the production stage or phase) and/or to optimize conditions during cell culture (e.g., concentrated feed media). Basal media formulations containing components essential for cell survival and growth are typically used to initiate a cell culture. Growth media formulations are typically used to promote cell growth and minimize protein expression. Production media formulations are typically used to promote production of the desired protein and maintain the cells, with minimal cell growth. A feed media, typically a concentrated media formulation to replenish nutrients and amino acids that are consumed during the production phase of the cell culture, may be used to supplement and maintain an active culture, particularly a culture operated in perfusion mode. Such a concentrated feed media may contain some, if not all, of the components of the cell culture media at, for example, about 5×, 6×, 7×, 8×, 9×, 10×, 12×, 14×, 16×, 20×, 30×, 50×, 100×, 200×, 400×, 600×, 800×, or 1000× of their normal amount or concentration.

A method of the present disclosure can be used as part of a larger production process whereby cells are cultured in distinct stages. Each stage can be conducted in its own bioreactor vessel or other vessel suitable for cell culture. Alternatively, more than one stage can be conducted in a common vessel. For commercial production of recombinant proteins by mammalian cells, there are commonly multiple, for example, at least about 2, 3, 4, 5, 6, 7, 8, 9, or 10 growth stages that occur in different culture vessels preceding the final N production stage. For example, cells may be cultured in various bioreactors for one or more growth stages prior to the N−1 seed stage, and then cultured in one or more N−1 bioreactors. The duration of the N−1 stage can range from, e.g., 3 to 14 days, or be continuous, and can be designed to maintain the cells in exponential growth prior to inoculation of a production (N) production bioreactor. The cells from the N−1 bioreactor may be transferred to a (N) production bioreactor and grown under conditions that maximize protein production.

As used herein, the term “bioreactor” refers to any vessel useful for the growth of a cell culture (e.g., a mammalian cell culture). Non-limiting examples of bioreactors include stirred tank, airlift, fiber, microfiber, hollow fiber, ceramic matrix, fluidized bed, fixed bed, and/or spouted bed bioreactors. Bioreactors can be single use and/or built-in-place stainless steel vessels. In some embodiments, an example bioreactor can perform one or more (e.g., one, two, three, all) of the following steps: feeding of nutrients and/or carbon sources, injection of suitable gas (such as, e.g., oxygen), inlet and outlet flow of cell culture medium, separation of gas and liquid phases, maintenance of temperature, maintenance of oxygen and CO₂ levels, maintenance of pH level, agitation (e.g., stirring), and/or cleaning/sterilizing. Any suitable bioreactor diameter can be used. Unless otherwise indicated by context, in some embodiments, the bioreactor can have a volume between 100 mL and 50,000 L. Unless otherwise indicated, a bioreactor can be of any size so long as it is useful for the culturing of cells; typically, a bioreactor is sized appropriate to the volume of cell culture being grown inside of it. In non-limiting embodiments and unless otherwise indicated by context, a bioreactor may be at least 1 liter (L) or may be 2, 5, 10, 50, 100, 200, 250, 500, 1,000, 1500, 2000, 2,500, 5,000, 8,000, 10,000, 12,000 liters or more, or any volume in between. In a preferred embodiment, the bioreactor is 200 to 20,000 liters. In one embodiment, the bioreactor volume is 2,000 liters to 18,000 liters. In one embodiment, the bioreactor volume is 2,000 liters to 15,000 liters. In one embodiment, the bioreactor volume is 2,000 liters to 12,000 liters. In one embodiment, the bioreactor volume is 2,000 liters to 10,000 liters. In one embodiment, the bioreactor volume is 200 liters to 5,000 liters. In one embodiment, the bioreactor volume is 2,000 liters to 3,000 liters. In one embodiment, the bioreactor volume is 10,000 liters to 20,000 liters. In one embodiment, the bioreactor volume is 10,000 liters to 18,000 liters. In one embodiment, the bioreactor volume is 10,000 liters to 15,000 liters. In one embodiment, the bioreactor volume is 10,000 liters to 12,000 liters. In one embodiment, the bioreactor volume is 200, 500, 1000, 2,000, 3,000, 5,000, 10,000, 12,000, 15,000, 18,000, or 20,000 liters.

The internal conditions of the bioreactor, including, but not limited to, pH and temperature, can be controlled during the culturing period. Those of ordinary skill in the art will be aware of, and will be able to select, suitable bioreactors for use in methods disclosed herein based on the relevant considerations.

The bioreactor volume is divided into the working volume space and the headspace. The working volume of the bioreactor refers to the volume within the bioreactor in which the cell culture is operated, typically expressed as a percentage of the bioreactor volume. In one embodiment, the working volume of the bioreactor is at least 70% of the bioreactor volume. In one embodiment, the working volume of the bioreactor is at least 70% to 100% of the bioreactor volume. In one embodiment, the working volume of the bioreactor is at least 75% of the bioreactor volume. In one embodiment, the working volume of the bioreactor is at least 80% of the bioreactor volume. In one embodiment, the working volume of the bioreactor is at least 85% of the bioreactor volume. In one embodiment, the working volume of the bioreactor is at least 90% of the bioreactor volume. In one embodiment, the working volume of the bioreactor is at least 91% of the bioreactor volume. In one embodiment, the working volume of the bioreactor is at least 92% of the bioreactor volume. In one embodiment, the working volume of the bioreactor is at least 93% of the bioreactor volume. In one embodiment, the working volume of the bioreactor is at least 94% of the bioreactor volume. In one embodiment, the working volume of the bioreactor is at least 95% of the bioreactor volume. In one embodiment, the working volume of the bioreactor is at least 96% of the bioreactor volume. In one embodiment, the working volume of the bioreactor is at least 97% of the bioreactor volume. In one embodiment, the working volume of the bioreactor is at least 98% of the bioreactor volume. In one embodiment, the working volume of the bioreactor is at least 99% of the bioreactor volume. In one embodiment, the working volume of the bioreactor is about 100% of the bioreactor volume.

In some embodiments, the lean perfusion method described herein is conducted using single-use bioreactors instead of traditional stainless steel culture vessels. Use of single-use technology minimizes infrastructure requirements associated with traditional cell culture, such as steel/glass commercial-scale vessels and associated machinery. Single-use bioreactors provide flexibility to the manufacturing process; site assembly, reconfiguration, sterilization, and validation of single-use bioreactors is often faster, easier, and less costly than traditional built-in-place stainless steel cell culture plants. Single use bioreactors comprise disposable, plastic sterile bags supported by a non-disposable support structure. The culture is agitated by a stirrer within the bag or by rocking, air and oxygen spargers are also supplied as well as sensors to measure and adjust various parameters of the culture, such as pH, temperature, oxygen, cell density, and the like. Single-use bioreactors are commercially available, for example, Bio STR®, Sartorius, Gottingen Germany; MOBIUS®, Millipore, Burlington, MA; XCELLEREX®, Cytiva, Marlborough, MA.

In some embodiments, a lean perfusion method disclosed herein is conducted in one or more stainless-steel bioreactors, particularly built-in-place large-scale stainless-steel bioreactors capable of operating at volumes of 2,000 to 20,000 liters or more.

The bioreactor system maintains conditions within the bioreactor to support cell culture. Suitable culture conditions for mammalian cells are known in the art. See e.g. Animal cell culture: A Practical Approach, D. Rickwood, ed., Oxford University Press, New York (1992). As used herein, “running” a bioreactor system refers to maintaining conditions in the bioreactor system to support cell culture. A bioreactor “run” typically comprises the steps of inoculating a prepared bioreactor with a seed culture, subjecting the cells to one or more growth phase and/or production phases until one or more predetermined parameters are met (time, viable cell density, packed cell volume) and then harvesting the contents of the bioreactor.

The harvesting operation fully or partially clarifies and/or purifies the target protein away from at least one impurity with which it is found in the cell culture fluid, such as remaining cell culture media, cells, cell debris, undesired cell, or media components, and/or product- and/or process-related impurities. Methods for harvesting recombinant proteins from suspension cell cultures are known in the art and include, but are not limited to, acid precipitation, accelerated sedimentation methods such as flocculation, separation using gravity, centrifugation, acoustic wave separation, filtration, including membrane filtration, ultrafilters, microfilters, tangential flow, alternative tangential flow, depth filters, and alluvial filtration filters.

Harvested cell culture fluid (HCCF) can be stored in surge tanks, holding tanks, bags, or other containers that are adapted to provide feed to a chromatography column skid and are appropriate for the infrastructure and/or process requirements.

The harvested cell culture fluid may be subjected to one or more downstream operations to capture and/or polish the target protein. The downstream operations make use of affinity chromatography, including resins and/or membranes containing agents that will bind and/or interact in some manner with at least a desired protein, impurity, or contaminant. Affinity chromatography is commonly used in biomanufacturing processes, typically as an initial capture step to isolate and concentrate desired proteins from harvested cell culture fluid. Non-limiting examples of such affinity chromatography materials include those that make use of Staphylococcus proteins such as Protein A, Protein G, Protein A/G, and Protein L; substrate-binding capture mechanisms; antibody- or antibody fragment-binding capture mechanisms; aptamer-binding capture mechanisms; cofactor-binding capture mechanisms; and the like. Immobilized metal affinity chromatography (IMAC) can be used to capture proteins that have or have been engineered to have affinity for metal ions. Protein A affinity chromatography is typically used in first-line, bulk purification operations. Protein A ligands are highly selective for a wide range of proteins containing an antibody Fc region and provide a robust removal of process-related impurities and high target protein yields. Protein A material is available commercially from a number of vendors, such as, for example, MABSELECT™ SURE Protein A, Protein A Sepharose FAST FLOW™, MABSELECT™ PrismA (Cytiva, Marborough, MA), PROSEP-A™ (Merck Millipore, U.K), TOYOPEARL™ HC-650F Protein A (TosoHass Co., Philadelphia, PA), and AP Plus, Purolite, King of Prussia, PA).

Intermediate and/or polishing unit operations make use of various chromatography methods for the continued purification of the desired protein and clearance of contaminants and impurities such as process- and product-related impurities, contaminants, virus, and the like. These chromatography unit operations make use of resins and/or membranes containing agents that can be operated in a variety of modes; two commonly used modes are bind-and-elute mode and flow-though mode. Frontal chromatography mode allows for a continuous, high-density feed (containing the protein of interest and at least one impurity) on to the chromatography medium. In frontal chromatography, the separation of the protein of interest from impurities and contaminants is driven by the binding affinity of the components in the load feed for the chromatography medium. The amount of the protein of interest that may be loaded on and bound to the chromatography medium in frontal mode is typically dependent on the amount of more highly charged impurities/contaminants, such as product-related impurities, in the load feed. Initially, all the components in the load feed will bind to the chromatography medium. Separation of the product of interest from the impurities/contaminants is driven by affinity for the chromatography medium. When the chromatography medium reaches saturation binding, those components in the load feed having a greater affinity for the chromatography medium (typically product-related impurities such as a HMW species) will displace proteins having a weaker affinity (the product of interest), resulting in the separation of the proteins with weaker affinity from the chromatography medium. These proteins exit the column in the load flow through as a pure band. As loading proceeds, the bound proteins are continuously displaced in order of increasing affinity for the chromatography medium until the column is at or near saturation with proteins having greater affinity than the protein of interest.

Examples of chromatography media used in such polishing processes include, but are not limited to, media for ion exchange chromatography (IEX), such as anion exchange chromatography (AEX) and cation exchange chromatography (CEX); hydrophobic interaction chromatography (HIC); mixed modal or multimodal chromatography (MM); and hydroxyapatite chromatography (HA).

Multiple purification chromatography unit operations (e.g., one, two, or three chromatography unit operations), each typically performing a different function or operating in a different mode, may be combined depending on the requirements of the manufacturing process. Cation exchange chromatography refers to chromatography performed on a solid phase medium (e.g., resin or membrane) that is negatively charged and has free cations for exchange with cations in an aqueous solution passed over or through the solid phase. The charge may be provided by attaching one or more charged ligands to the solid phase, e.g., by covalent linking. Alternatively, or in addition, the charge may be an inherent property of the solid phase (e.g., as is the case for silica, which has an overall negative charge). CEX chromatography is typically used to remove high molecular weight (HMW) contaminants, process related impurity, and/or viral clearance. Commercially available cation exchange media include, but are not limited to, sulphopropyl (SP) immobilized on agarose (e.g., SP-SEPHAROSE FAST FLOW™, SP SEPHAROSE FAST FLOW XL™ or SP-SEPHAROSE HIGH PERFORMANCE™, CAPTO S™ CAPTO SP ImpRes™, CAPTO S ImpAct™ (Cytiva), FRACTOGEL-SO3™, FRACTOGEL-SE HICAP™, and FRACTOPREP™ (EMD Merck, Darmstadt, Germany), TOYOPEARL® XS, TOYOPEARL® HS (Tosoh Bioscience, King of Prussia, PA), UNOsphere™ (BioRad, Hercules, CA), S Ceramic Hyper™ DF (Pall, Port Washington, NY), POROS™ (ThermoFisher, Waltham, MA), ESHMUNO® CSP and ESHMUNO® CP-FT (Millipore Sigma, Darmstadt, Germany).

Anion exchange chromatography refers to chromatography performed on a solid phase medium (e.g., resin or membrane) that is positively charged and has free anions for exchange with anions in an aqueous solution passed over or through the solid phase. AEX chromatography is used, for example, for viral clearance and impurity removal. Commercially available anion exchange media include, but are not limited to, sulphopropyl (SP) immobilized on agarose (e.g. Source 15 Q, Capto™ Q, Q-SEPHAROSE FAST FLOW™ (Cytiva), FRACTOGEL EDM TMAE™, FRACTOGEL EDM DEAE™ (EMD Merck), TOYOPEARL Super Q® and TOYOPEARL NH2-750F (Tosoh Bioscience), POROS HQ™, POROS XQ™, (ThermoFisher).

Mixed-mode or multi-mode chromatography (MMC) refers to chromatography that makes use of more than one form of interaction between the stationary phase and analyte to achieve their separation. MMC differs from single mode chromatography in that two or more types of interactions, such as electrostatic, hydrogen bonding and hydrophobic interactions, contribute significantly to the retention of solutes. Commercially available multi-modal chromatography media include, but are not limited to, Capto™ Adhere, Capto™ MMC Impress, Capto MMC, (Cytiva), PPA Hypercel, MEP Hypercell, HEA Hypercell (Pall Corporation, Port Washington, NY). Eshmuno HCX, (Merk Millipore), Toyopearl MX-Trp-650M (Tosoh Bioscience).

Hydrophobic interaction chromatography refers to chromatography performed on a solid phase medium that makes use of the interaction between hydrophobic ligands and hydrophobic residues on the surface of a target protein. Commercially available hydrophobic interaction chromatography media include, but are not limited to, Phenyl Sephrose™ (Cytiva), Tosoh Hexyl (Tosoh Bioscience), and Capto™ Phenyl (Cytiva).

Hydroxyapatite chromatography refers to chromatography performed on a solid phase medium that makes use of positively charged calcium and negatively charged phosphate and depending on the pI of the protein and the pH of the buffer, can act as a cation or anion. Commercially available hydroxyapatite media include, but are not limited to, CA⁺⁺Pure-HA, Tosoh Bioscience, HA ULTROGEL®, Sartorius).

Unit operations directed towards inactivating, reducing, and/or eliminating viral contaminants may include processes that mitigate viral risk by manipulating the environment and/or through use of filtration. Viral mitigation measures are critical to ensure the safety of protein therapeutics and may be performed one or more times throughout the downstream purification. Viral contaminants can arise from a variety of sources, including use of reagents of animal origin, adventitious viral contaminants in host cell lines, or system failures at GMP manufacturing sites. Viruses are classified as enveloped and non-enveloped viruses. With enveloped viruses, the envelope allows the virus to identify, bind, enter, and infect target host cells. As such, enveloped viruses are susceptible to inactivation methods. Various methods can be employed for virus inactivation and include, but are not limited to, heat inactivation/pasteurization, UV and gamma ray irradiation, use of high intensity broad spectrum white light, addition of chemical inactivating agents, surfactants, and solvent/detergent treatments. Surfactants, such as detergents, solubilize membranes and therefore can be very effective in specifically inactivating enveloped viruses. One method for achieving virus inactivation is incubation at low pH (e.g., pH<4). Low pH virus inactivation can be followed by neutralization that re-adjusts the viral inactivated solution to a pH more compatible with the requirements of the following downstream unit operations. Low pH viral inactivation is typically performed following purification of the harvest cell culture fluid with affinity chromatography, in particular affinity chromatography that makes use of a substrate binding ligand from Staphylococcus aureus, such as Protein A chromatography, since elution is usually performed at a low pH. Non-limiting example low pH viral inactivation methods are described in U.S. Application No. 63/168,608 and U.S. Application No. 63/159,217. A non-limiting example of a detergent inactivation method is described in International Publication No. WO 2020/190985. Low pH viral inactivation may also be followed by filtration, such as depth filtration, for clarification of the neutralized fluid.

Non-enveloped viruses are more difficult to inactivate without risk to the protein being manufactured and are removed by filtration methods. An exemplary process is described in International Publication No. WO2020/159838. Viral filtration can be performed using micro- or nano-filters, such as those available from PLAVONA® (Asahi Kasei, Chicago, IL), VIROSART® (Sartorius, Goettingen, Germany), VIRESOLVE® Pro (MilliporeSigma, Burlington, MA), Pegasus™ Prime (Pall Biotech, Port Washington, NY), CUNO Zeta Plus VR, (3M, St. Paul, Mn). Viral filtration may occur at one or more steps in the downstream operations of a biomanufacturing process. Viral inactivation and viral filtration can take place at one or more stages in a downstream process. Typically viral filtration follows an affinity chromatography unit operation and viral filtration precedes or follows a ultrafiltration/diafiltration (UF/DF) operation but may also take place following UF/DF.

Downstream unit operations may also comprise product concentration and buffer exchange of the desired protein into a desired formulation buffer. A UF/DF operation may take place at one or more stages in a downstream process. Typically, a UF/DF operation is performed prior to bulk storage of the drug substance. Instead of storage, unit operations related to drug product fill/finish can also immediately follow a UF/DF operation. One or more stability-enhancing excipients may optionally be added directly to the UF/DF retentate feed tank containing the formulated purified protein resulting in formulated drug substance or added to the UF/DF eluate pool. An exemplary UF/DF process is described in International Publication No. WO 2020/159838. Filters for use in a UF/DF operation are well-known, common in the art, and commercially available from many sources, such as, e.g., regenerated cellulose Pellicon (MilliporeSigma, Danvers, MA), stabilized cellulose, Sartocon® Slice, Sartocon® ECO Hydrosart® (Sartorius, Goettingen, Germany), polyethersulfone (PES) membrane, Omega (Pall Corporation, Port Washington, NY).

In all chromatography processes, multiple filters can be used to the capacity that holders, skids, or the physical set up of the UF/DF system will allow or are needed to achieve the desired objectives of a production process.

The upstream and/or downstream unit operations can be performed in a continuous or stepwise manner. Vessels, such as surge vessels, storage tanks, and the like can be used following one or more unit operation.

Critical attributes and performance parameters of the purified desired protein can be measured to better inform decisions regarding performance of each step during manufacture. These critical attributes and parameters can be monitored real-time, near real-time, and/or following a unit operation. Critical parameters that can be measured during cell culture include, but are not limited to, cell culture media components that are consumed (such as, e.g., glucose), levels of metabolic by-products (such as, e.g., lactate and ammonia) that accumulate, as well as parameters related to cell maintenance and survival, such as, e.g., dissolved oxygen content. Critical attributes such as specific productivity, viable cell density, packed cell volume, pH, osmolality, appearance, color, aggregation, percent yield, and titer may also be monitored during appropriated stages in the manufacturing process. Monitoring and measurements can be performed using known techniques and commercially available equipment.

The lean perfusion cell culture methods described herein can be used to product polypeptides and proteins of interest. The polypeptides and proteins can be of scientific or commercial interest, including protein-based therapeutics. Proteins of interest include, but are not limited to, secreted proteins, non-secreted proteins, intracellular proteins, or membrane-bound proteins. Polypeptides and proteins of interest can be produced by recombinant animal cell lines using cell culture methods described herein and may be referred to as “recombinant proteins.” The expressed protein(s) may be produced intracellularly or secreted into the culture medium from which it can be recovered and/or collected. The term “isolated protein” or “isolated recombinant protein” refers to a polypeptide or protein of interest, that is purified away from proteins or polypeptides or other contaminants that would interfere with its therapeutic, diagnostic, prophylactic, research, or other use. Proteins of interest include, but are not limited to, proteins that exert a therapeutic effect by binding a target, such as, e.g., a target among those listed below, including targets derived therefrom, targets related thereto, and modifications thereof.

Proteins of interest may include, but are not limited to, “antigen-binding proteins.” An “antigen-binding protein” refers to a protein or polypeptide that comprises an antigen-binding region or antigen-binding portion that has affinity for another molecule to which it binds (antigen). Antigen-binding proteins include, but are not limited to, antibodies, peptibodies, antibody fragments, antibody derivatives, antibody analogs, fusion proteins (including, e.g., single-chain variable fragments (scFvs), double-chain (divalent) scFvs, and IgGscFv (see, e.g., Orcutt et al., 2010, Protein Eng Des Sel 23:221-228)), hetero-IgG (see, e.g., Liu et al., 2015, J Biol Chem 290:7535-7562), muteins, and XmAb® (Xencor, Inc., Monrovia, CA). Also included are BiTE® molecules, bispecific T cell engagers, bispecific T cell engagers having extensions, and others, chimeric antigen receptors (CARs, CAR Ts), and T cell receptors (TCRs).

As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e., between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

As used in the context of this disclosure, an “antigen-binding fragment,” used interchangeably herein with “binding fragment” or “antibody fragment,” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g., VH domain of heavy chain only antibodies (e.g., camelid heavy chain antibodies); VHH fragment, see Cortez-Retamozo et al., Cancer Research, Vol. 64:2853-57, 2004), a Fab fragment, a Fab′ fragment, a F(ab′)₂ fragment, a Fv fragment, a Fd fragment, and a CDR fragment, and can be derived from any mammalian source, such as human, mouse, rat, rabbit, or camelid. Antigen-binding fragments may compete for binding of a target antigen with an intact antibody, and the fragments may be produced by the modification of intact antibodies (e.g., enzymatic or chemical cleavage) or synthesized de novo using recombinant DNA technologies or peptide synthesis. In some embodiments, the antigen-binding fragment comprises at least one CDR from an antibody that binds to the antigen, for example, the heavy chain CDR3 from an antibody that binds to the antigen. In other embodiments, the antigen-binding fragment comprises all three CDRs from the heavy chain of an antibody that binds to the antigen or all three CDRs from the light chain of an antibody that binds to the antigen. In still other embodiments, the antigen-binding fragment comprises all six CDRs from an antibody that binds to the antigen (three from the heavy chain and three from the light chain).

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc domain” of an immunoglobulin generally comprises two constant domains, a CH2 domain and a CH3 domain, and optionally comprises a CH4 domain. The Fc domain may be an Fc domain from an IgG1, IgG2, IgG3, or IgG4 immunoglobulin. In some embodiments, the Fc domain comprises CH2 and CH3 domains from a human IgG1 or human IgG2 immunoglobulin. The Fc domain may retain effector function, such as C1q binding, complement dependent cytotoxicity (CDC), Fc receptor binding, antibody-dependent cell-mediated cytotoxicity (ADCC), and phagocytosis. In other embodiments, the Fc domain may be modified to reduce or eliminate effector function.

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies, with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure. Human heavy-chain antibodies can be produced by transgenic animals expressing human immunoglobulin genes, such as UniAb™ antibodies produced by UniRat™ transgenic rats.

In some embodiments, proteins of interest may include colony stimulating factors, such as, e.g., granulocyte colony-stimulating factor (G-CSF). Such G-CSF agents include, but are not limited to, Neupogen® (filgrastim) and Neulasta® (pegfilgrastim). Also included are erythropoiesis stimulating agents (ESA), such as, e.g., Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, epoetin omega, epoetin iota, tissue plasminogen activator, and GLP-1 receptor agonists, as well as variants or analogs thereof and biosimilars of any of the foregoing.

In some embodiments, proteins of interest bind to one of more of the following, alone or in any combination: CD proteins including, but not limited to, CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including, but not limited to, insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGβ, hepatitis-C virus, mesothelin dsFv[PE38] conjugate, Legionella pneumophila (lly), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40 L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In some embodiments, proteins of interest include abciximab, adalimumab, adecatumumab, aflibercept, alemtuzumab, alirocumab, anakinra, atacicept, basiliximab, belimumab, bemarituzumab, bevacizumab, biosozumab, blinatumomab, brentuximab vedotin, brodalumab, cantuzumab mertansine, canakinumab, cetuximab, certolizumab pegol, conatumumab, daclizumab, denosumab, eculizumab, edrecolomab, efalizumab, epratuzumab, erenumab, etanercept, evolocumab, galiximab, ganitumab, gemtuzumab, golimumab, ibritumomab tiuxetan, infliximab, ipilimumab, lerdelimumab, lumiliximab, lxdkizumab, mapatumumab, motesanib diphosphate, muromonab-CD3, natalizumab, nesiritide, nimotuzumab, nivolumab, ocrelizumab, ofatumumab, omalizumab, oprelvekin, ordeskimab, palivizumab, panitumumab, pembrolizumab, pertuzumab, pexelizumab, ranibizumab, rilotumumab, rituximab, romiplostim, romosozumab, sargramostim, tocilizumab, tositumomab, tarlatamab, trastuzumab, ustekinumab, vedolizumab, visilizumab, volociximab, zanolimumab, and zalutumumab, as well as biosimilars of any of the foregoing.

In some embodiments, a recombinant protein produced by a method described herein is selected from bemarituzumab, denosumab, erenumab, evolocumab, ordeskimab, panitumumab, romosozumab, and tarlatamab.

In some embodiments, proteins of interest can also include genetically engineered receptors, such as, e.g., chimeric antigen receptors (CARs) and T-cell receptors (TCRs), as well as other proteins comprising an antigen binding molecule that interacts with that targeted antigen. CARs can be engineered to bind to an antigen (such as, e.g., a cell-surface antigen) by incorporating an antigen-binding molecule that interacts with that targeted antigen. CARs typically incorporate an antigen binding domain (such as scFv) in tandem with one or more costimulatory (“signaling”) domains and one or more activating domains.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

Throughout this specification and the claims which follow, unless the context requires otherwise, the term “comprise,” and variations such as “comprises” and “comprising,” will be understood to imply the inclusion of a stated integer or step or group of integers or steps but not the exclusion of any other integer or step or group of integer or step. When used herein, the term “comprising” can be substituted with the term “containing” or “including” or sometimes when used herein with the term “having”.

As used herein, “consisting of” excludes any element, step, or ingredient not specified in the embodiment feature or claim element. As used herein, “consisting essentially of” does not exclude materials or steps that do not materially affect the basic and novel characteristics of the embodiment feature or claim element.

In each instance herein, any of the terms “comprising,” “consisting essentially of,” “consisting of,” and their variations may be replaced with any of the other two terms or their variations.

In some embodiments, the term “about” as used herein and as applied to one or more values refers to a value that is similar to a stated reference value. For example, in some embodiments, the term “about” refers to a range of values that fall within 10%, 9%, 8%, 7%, 6%, 5%, 4%, 3%, 2%, or 1%, or less in either direction (greater than or less than) of the stated reference value unless otherwise stated or otherwise evident from the context (e.g., except where such number would exceed 100% of a possible value).

All documents, or portions of documents, cited in this application, including but not limited to patents, patent applications, articles, books, and treatises, are hereby expressly incorporated by reference.

What is described in one embodiment of the disclosure can be combined with one or more other embodiments of the disclosure unless context clearly indicates otherwise. For example, unless context clearly indicates otherwise, recitations of “in one embodiment” in this specification can be replaced by “in some embodiments” or “in any embodiment.”

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawings. Such modifications are intended to fall within the scope of the disclosed subject matter.

The descriptions of the various embodiments and/or examples of the disclosed subject matter have been presented for purposes of illustration but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and/or to enable others of ordinary skill in the art to understand the disclosed subject matter.

### EXAMPLES

### Example 1 a Fed Batch Cell Culture Compared to a Lean Perfusion Cell Culture

### Fed Batch

Three CHO cell lines, each expressing a different monoclonal antibody (mAb 1, mAb 2, and mAb 3), were cultured under fed batch conditions. Each cell line was inoculated at about 0.8×10⁶ cells/mL to 1.2×10⁶ cells/mL into separate 3-liter bioreactors containing approximately 1.5 liters of serum free, chemically defined culture medium (n=3-16 per mAb). The bioreactors were maintained at 36° C., 5% CO₂, 6.90 pH, and 315 rpm. The cultures were fed a nutrient feed on days 3 and 6 at 8% post-inoculation volume, and a supplement feed on the same days at 4% of the nutrient feed volume. A glucose solution was added as needed to maintain the glucose concentration at >2 g/L during the entire culture. The mAb 2 and mAb 3 cultures were harvested on day 11, and the mAb 1 cultures were harvested on day 12.

### Lean Perfusion

The same three CHO cell lines expressing mAb 1, mAb 2, or mAb 3 were each inoculated at 6×10⁶ cells/mL to 12×10⁶ cells/mL into separate 7-liter bioreactors at an initial culture volume of about 3 liters of serum free, chemically defined basal culture medium (n=2-8 per mAb). The bioreactors were maintained at 36° C., 5% CO₂, 6.90 pH, and 315 rpm. Perfusion was initiated on day 1 (or about 24 hours post-inoculation) and used an alternating tangential flow filter system (Repligen, Waltham, MA) with a 30,000 MWCO filter (Cytiva, Westborough, MA). The cultures were perfused with a serum free, chemically defined feed medium. The feed rate and permeate rate were each maintained at 0.13 culture volumes/day (V/d). On day 3, a feed up phase was initiated to increase the culture volume in the bioreactors to a final culture volume of about 4.4 liters, about 100% of the final working volume of the bioreactor. The feed rate was increased to 0.22 V/d and the permeate rate was held constant at 0.13 V/d. On day 4, the feed up phase ended, and the feed rate was decreased to 0.13 V/d, the same as the permeate rate. The viable cell density following the feed up phase was about (20-30)×10⁶ cells/mL. The temperature of the culture was decreased to 33.0° C. within 48 hours post-feed up. On day 5, the feed rate and permeate rate were increased to 0.22 V/d until harvest. The mAb 1, mAb 2 and mAb 3 cultures were all harvested on day 15.

In-process samples were taken to monitor culture conditions. Viable cell density (VCD) refers to the number of live cells in a given volume of culture medium. VCD was determined using a Vi-Cell XR Cell Viability Analyzer (Beckman Coulter, Indianapolis, IN). Titer was measured by high performance liquid chromatography (HPLC) via affinity chromatography (Protein A, Waters, Milford, MA).

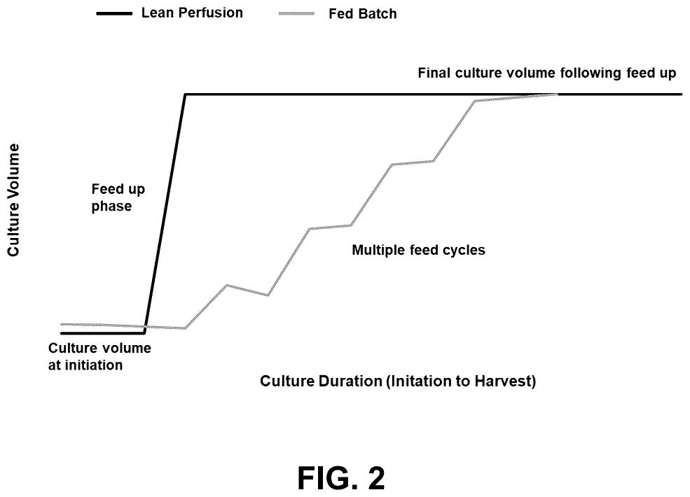

FIG. 2 shows a schematic representation of the change in culture volume over the course of cell culture from initiation to harvest. A typical lean perfusion culture with a single feed up phase (black line) is compared to a typical fed batch culture with multiple feeds (gray line). Both cultures are initiated at a culture volume of at least 50% to 75% of their respective final working volumes. The lean perfusion method makes use of a shortened growth phase at a low culture volume. When the culture reaches one or more desired target criteria, such as cell density or time post-inoculation, the culture volume is increased to the desired final culture volume (typically greater than 75% to 100% of the final working volume). The production phase is initiated, enabling the lean perfusion method to make efficient use of the maximum working volume of the bioreactor for a longer period during the culture operation, compared with the fed batch process which slowly increases the culture volume stepwise or intermittently and is only able to make use of the maximum working volume of the bioreactor at the end of the culture. Achieving the desired final culture volume and cell density early in the cell culture process leads to increased production with the lean perfusion culture compared to the fed batch culture.

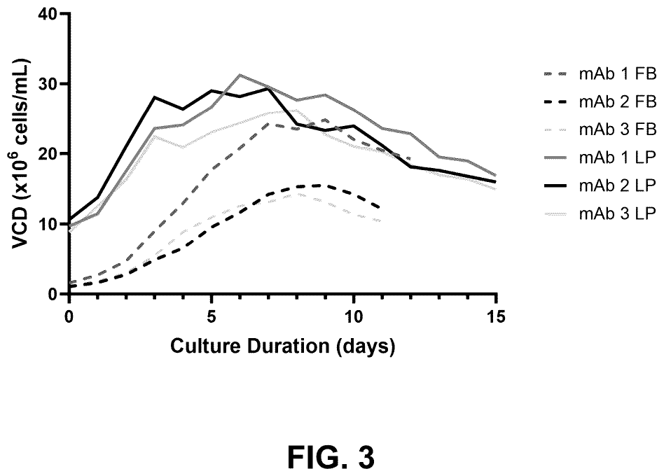

FIG. 3 shows the viable cell densities achieved by the lean perfusion cultures and fed batch cultures for the three monoclonal antibody samples. The lean perfusion cultures made use of high cell densities in low culture volumes to achieve and maintain a higher viable cell density during the growth phase as compared to the fed batch cultures. Lean perfusion: mAb 1 solid dark gray line, mAb 2 solid black line, mAb 3 solid light gray line. Fed batch: mAb 1 dashed dark gray line, mAb 2 dashed black line, mAb 3 dashed light gray line. The lean perfusion cultures incorporated a single feed up phase, which increased the culture volume and reduced the cell mass prior to the production phase. During the production phase, the lean perfusion culture maintained a low perfusion rate to provide feed media and remove waste to achieve a longer culture duration compared to the fed batch culture. In addition, although both cultures were initiated at about 50% of the final culture volume, the lean perfusion samples were able to support a 10× increase in cell density at inoculation compared to the fed batch samples.

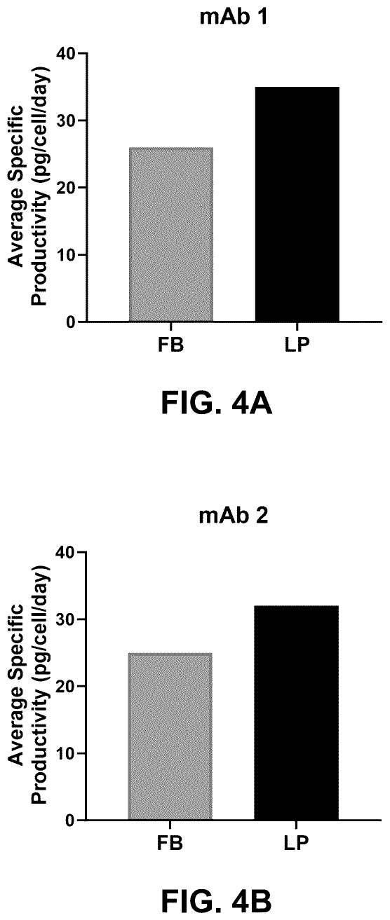

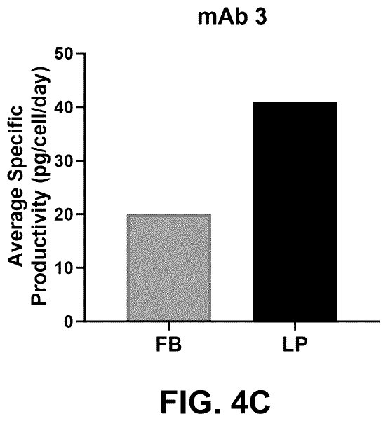

FIGS. 4A-4C show the average specific productivity (pg/cell/day) for each culture type: lean perfusion culture (LP), fed batch culture (FB). The specific productivity of the lean perfusion cultures showed an increase compared to the fed batch cultures, 27% with mAb 1, 22% with mAb 2, and 510% with mAb 3.

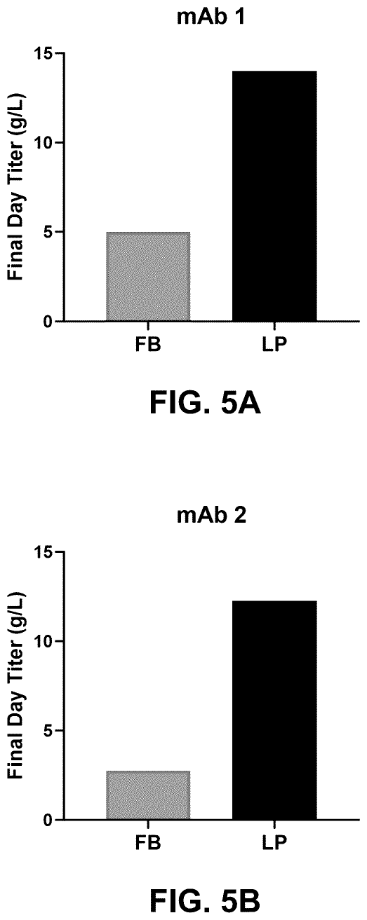

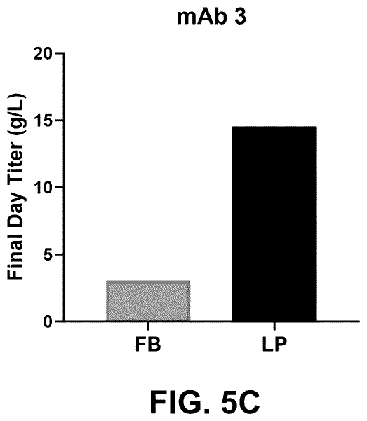

FIGS. 5A-5C show the average final day titer (g/L) for each culture type. The lean perfusion samples showed an increase in final day titer compared to their fed batch counterparts: mAb 1 showed a 3× increase, mAb 2 showed a 4.4× increase, and mAb 3 showed a 4.7× increase.

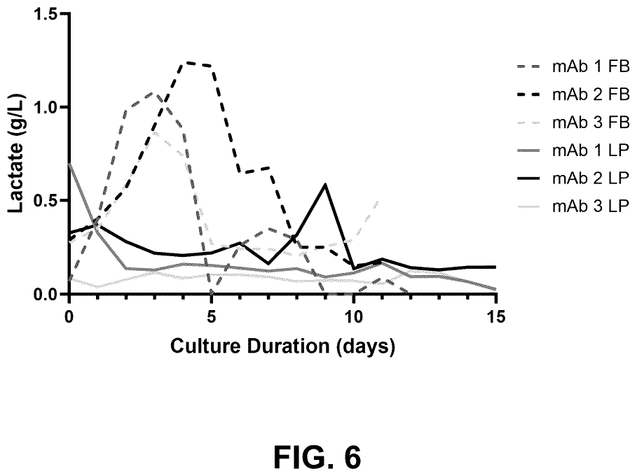

FIG. 6 shows a comparison of lactate concentration for the lean perfusion cultures and the fed batch cultures. The low perfusion rates used in the lean perfusion culture process reduced cell culture waste buildup by removing spent media in the permeate flow, as shown by the reduction in lactate concentration throughout the culture duration compared to the fed batch process, which simply diluted the lactate in the culture volume.

The lean perfusion culture method made efficient use of the bioreactor working volume during the entire culture duration. Inoculating the bioreactor containing a low cell culture medium volume at a high cell density enabled a shortened growth phase, allowing most of the culture time to be spent in production at a desired cell density. Also, increasing the culture volume prior to production lowered the cell mass which reduced accumulation of impurities, such as lactate. In addition, a low perfusion rate decreased the quantity of feed media required to maintain the culture during both the growth and production phases. As a result, the productivity was increased compared to the fed batch culture. In a fed batch process, the volume and cell density are slowly increased over the course of the culture, only achieving full capacity when the culture begins to decline and is harvested.

### Example 2 a Perfusion Culture (High Perfusion Rate, Constant Culture Volume) Compared to a Lean Perfusion Cell Culture

### Perfusion Culture

Two CHO cell lines (mAb 2 and mAb 3) were each inoculated at about 0.5×10⁶ cells/mL to 2×10⁶ cells/mL into separate 3-liter bioreactors in 1.5 liters of serum free, chemically defined basal culture medium, about 100% of the final working volume of the bioreactor (n=3-8 per mAb). The bioreactors were maintained at 36° C., 5% CO₂, 6.90 pH, and 350 rpm. Perfusion was initiated on day 3 and used an alternating tangential flow filter system (Repligen) with a 30,000 MWCO filter (Cytiva). The cultures were perfused with a serum free, chemically defined feed medium. At initiation, the feed rate and permeate rate were 0.5 V/d. On day 5, the feed rate and permeate rate were increased to 0.75 V/d. On day 8, feed rate and permeate rate were increased to 1.0 V/d until harvest on day 15.

### Lean Perfusion

The same CHO cell lines (mAb 2 and mAb 3) were each inoculated at about 6×10⁶ to 12×10⁶ cells/mL into separate 7 L bioreactors at an initial culture volume of approximately 3 liters of serum free, chemically defined basal culture medium (n=2-8 per mAb). The bioreactors were maintained at 36° C., 5% CO₂, 6.90 pH, 315 rpm. Perfusion was initiated on day 1 (or about 24 hours post-inoculation) and used an alternating tangential flow filter system (Repligen, Waltham, MA) with a 30,000 NMWC filter (GE Healthcare, Westborough, MA). The cultures were perfused with a serum free, chemically defined feed medium. The feed rate and permeate rate were 0.13 V/d starting 24 hours post-inoculation. On day 3, a feed up period was initiated to increase the culture volume in the bioreactors to a final culture volume of about 4.4 L, about 100% of the final working volume of the bioreactor. The feed rate was increased to 0.386 V/d, and the permeate rate was held constant at 0.13 V/d in order to reach the full bioreactor volume in about 30 hours. On day 4, the feed up period ended, and feed rate decreased to 0.13 V/d, the same as the permeate rate. The temperature was decreased to 33.0° C. on day 5. The viable cell density at the end of the feed up period was about 30×10⁶ cells/mL. On day 5, the feed rate and permeate rate were increased to 0.22 V/d until harvest on day 15.

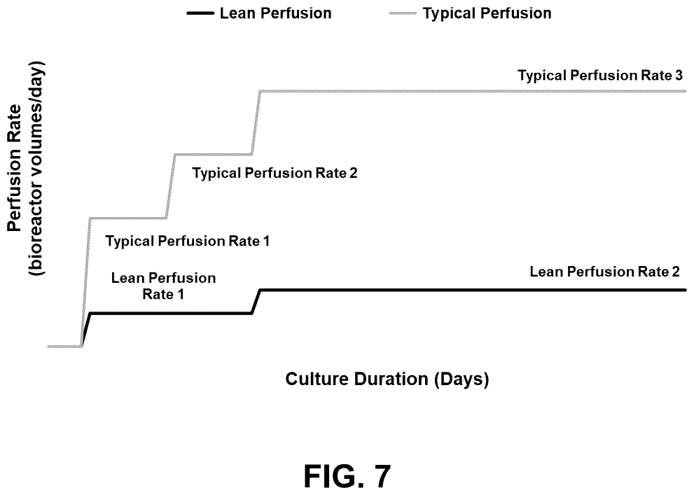

FIG. 7 shows a schematic representation of the different perfusion rates for the lean perfusion process (black line) compared to the high perfusion rate, high cell density prefusion process (gray line). The lean perfusion culture, like a typical perfusion culture process, relies on perfusion rates where the feed and permeate rates are the same, except during a feed up phase, where the feed rate is increased relative to the permeate rate to generate a net increase in culture volume in the bioreactor. The lean perfusion method makes use of low perfusion rates of less than or equal to 0.5 V/d throughout the cell culture process, preferably less than or equal to 0.25 V/d. The perfusion rate may be lower prior to the feed up phase (Rate 1) and the same or higher following the feed up phase (Rate 2). For a perfusion culture method, the rate is typically conducted at 1.0 V/d or more. Some perfusion culture methods make use of one or more quick incremental rate increases early in the culture, typically at least 0.5 V/d or higher, finishing at a rate of equal to or greater than 1.0 V/d. Making use of a high perfusion rate over the course of the culture operation means preparation, storage and use of higher volumes of feed media as well as an increased volume of spent media waste, with increased disposal and environmental costs.

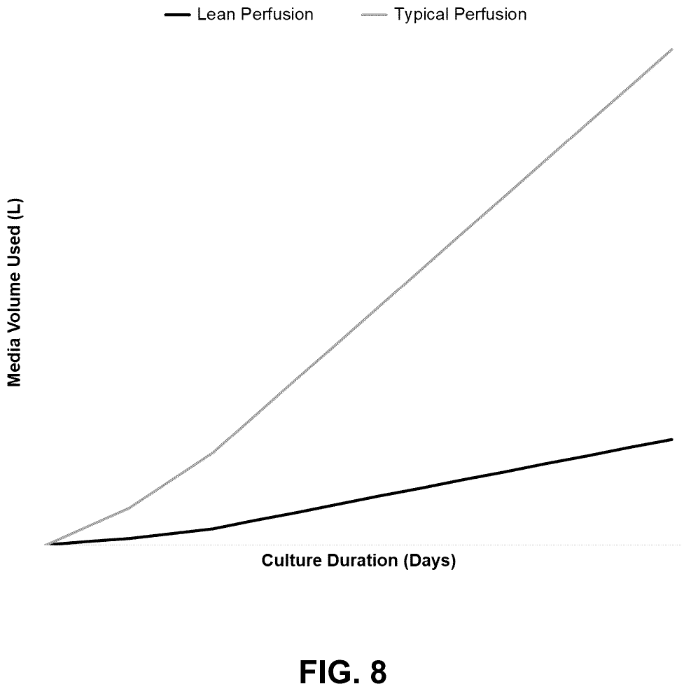

FIG. 8 shows a schematic representation of the magnitude of feed media used by the lean perfusion process (black line) which maintains a low-level consumption through the course of the culture, in contrast to a high perfusion rate process (gray line) which uses feed media at a rate of 2 or more times the rate of the lean perfusion process.

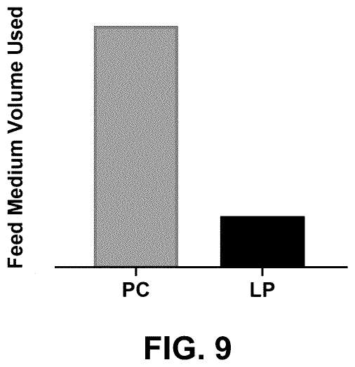

FIG. 9 shows a schematic representation of magnitude of the difference in the amount of feed media required for a lean perfusion culture (LP) black column and a perfusion culture operated at a high perfusion rate (PC) dark gray column. The low perfusion rate(s) and the initial low culture volume used in the lean perfusion method reduce the volume of feed media required, in some cases up to 4 times the amount compared to the perfusion culture which continuously operated at a higher perfusion rate and at a volume equal to the maximum working volume of the bioreactor.

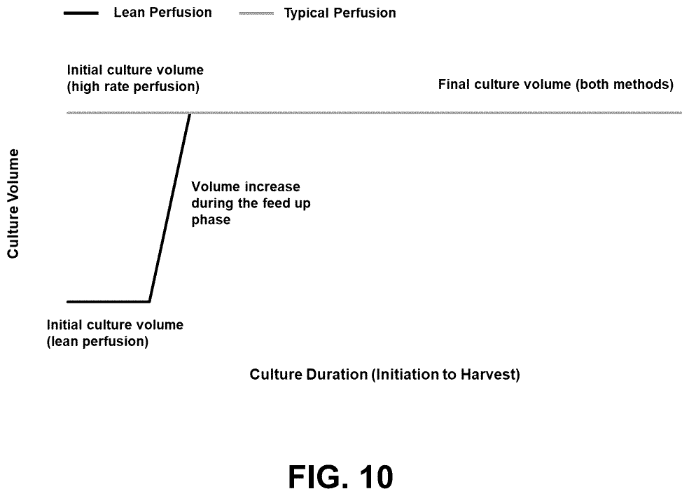

FIG. 10 shows a schematic representation of the difference in culture volumes over time for the lean perfusion culture (black line) compared to the high perfusion rate culture (gray line). The initial culture volume for lean perfusion was set at to at least 50% to 75% of the working volume of the bioreactor and increased to up to 100% of the working volume during the feed up phase, compared to a high-density perfusion culture, which operates at 100% of the bioreactor working volume for the duration of the culture. Starting with a lower culture volume allows for higher seeding from an N−1 reactor with a smaller working volume.

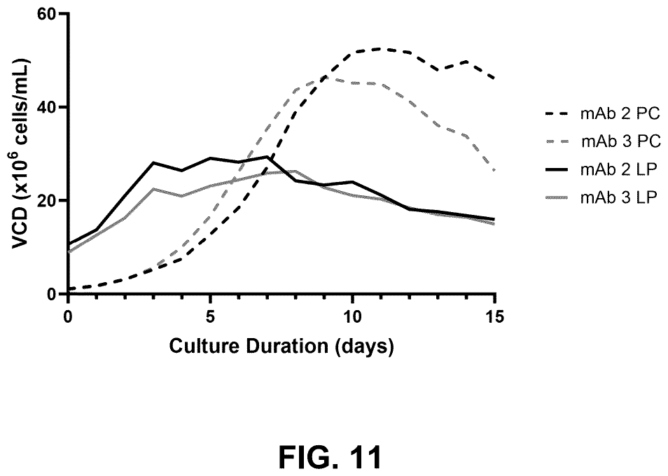

FIG. 11 shows a comparison of the viable cell density (VCD) of the lean perfusion cultures and the perfusion cultures operated at high perfusion rate. Lean perfusion culture: mAb 2 (solid black line), mAb 3 (solid gray line). Perfusion culture: mAb 2 (dashed black line), mAb 3 (dashed gray line). The lean perfusion culture was initiated at a high cell density in a culture volume that was less than 60% of the bioreactor working volume, which was increased to about 100% of the final working volume by the end of the feed up phase and maintained at that volume until harvest. The high perfusion rate culture was initiated at about 0.5×10⁶ cells/mL to 2×10⁶ cells/mL in 100% of the final bioreactor working volume. The cell density increased slowly to a viable cell density greater than that of the lean perfusion samples by the end of the culture. By initiating the culture at a low culture volume and high cell density, the lean perfusion culture achieved target cell densities more quickly, reducing the time required for the growth phase and maximizing time for production.

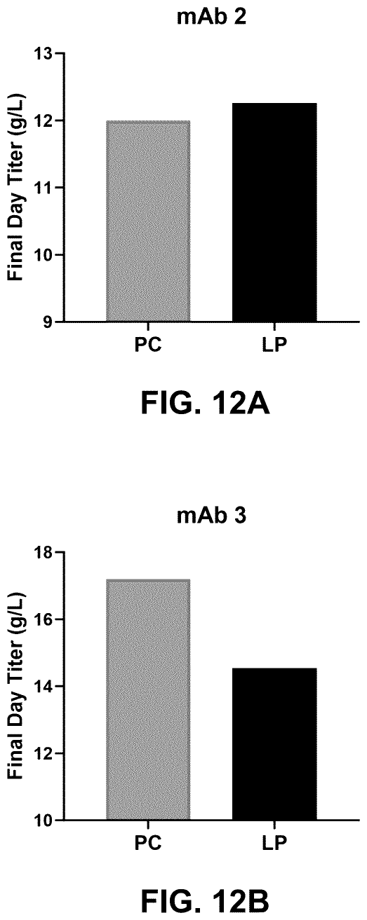

FIGS. 12A and 12B show the final day titer for the lean perfusion culture (LP, black column) compared to the perfusion culture operated at high perfusion rate (PC, gray column). The lean perfusion culture had comparable final titer to the perfusion culture. For mAb 2, there was an increase in titer of 1.1× for lean perfusion culture compared to the perfusion culture. For mAb 3, there was a decrease of 0.8× for the lean perfusion culture compared to the perfusion culture.

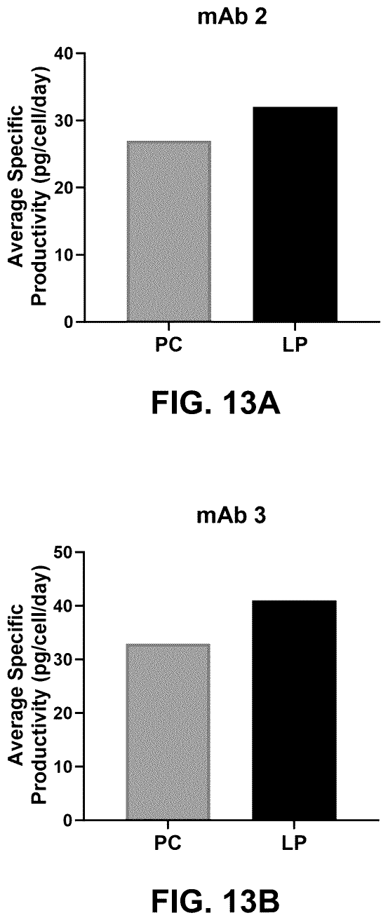

FIGS. 13A and 13B show the average specific productivity (pg/cell/day) over the course of the culture duration for the lean perfusion culture (LP, black column) and the perfusion culture operated at high perfusion rate (PC, gray column). Even though the lean perfusion culture operated at a lower perfusion rate and used less feed media compared to the high perfusion rate perfusion culture, the cells produced more protein of interest. Specific productivity with lean perfusion showed an increase of 18% with mAb 2 and an increase of 19% with mAb 3 as compared to the high perfusion rate culture.

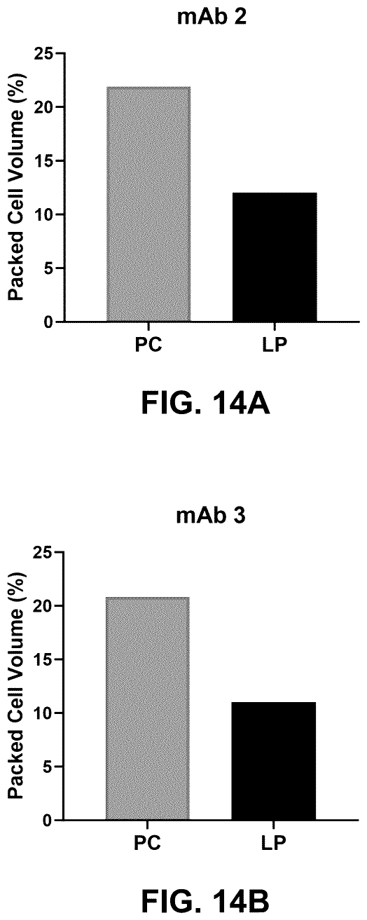

FIGS. 14A and 14B show the packed cell volume (%) of the lean perfusion culture compared to the high perfusion rate culture. Lean perfusion culture (LP): solid black column, Perfusion culture (PC): solid gray column. Lean perfusion packed cell volume showed a decrease of 45% with mAb 2 (FIG. 14A) and a decrease of 50% with mAb 3 (FIG. 14B) as compared to the high perfusion rate, high cell density perfusion culture.

Again, the lean perfusion culture method made efficient use of the low culture volume during the growth phase by maintaining a high cell density at a low perfusion rate. The lower culture volume and low perfusion rate allowed for the use of less feed media during the growth phase, compared to the high perfusion rate, high culture volume perfusion culture. The feed up phase increased the culture volume and lowered the cell density for the production phase, which again reduced the feed required and the waste produced compared to the high perfusion rate, high culture volume perfusion culture. Having a feed up period as a transition between the growth and production phases reduced the cell density and provided a large volume of fresh media to start the production phase. The perfusion rates were sufficient to remove waste while replenishing fresh nutrients to maintain cell productivity throughout the culture run. The temperature shift following the feed up period helped maintain the desired cell density for the production phase. In addition, although the packed cell volume was decreased up to 50%, the lean perfusion culture produced equivalent or higher final titers and specific productivity compared to the high perfusion rate, higher cell density, culture method.

### Example 3 a Fed Batch Cell Culture Compared to a Lean Perfusion Cell Culture

### Fed Batch

A CHO cell line expressing a monoclonal antibody (mAb 4) was cultured under fed batch conditions (n=5). The cell line was inoculated at about 10⁶ cells/mL into 3-liter bioreactors containing approximately 1.5 liters of serum free, chemically defined culture medium. The bioreactors were maintained at 36° C., 6.90 pH, and 350 rpm. The cultures were fed a nutrient feed on days 3, 6, 8, and 11 at 7%, 9%, 9%, and 9% of the post-inoculation volume, respectively, and a supplement feed on the same days at 4% of the nutrient feed volume. A glucose solution was added as needed to maintain the glucose concentration at >2 g/L during the entire culture. The mAb 4 cultures were harvested on day 13.

### Lean Perfusion

The same CHO cell line expressing mAb 4 was inoculated at about 75×10⁵ cells/mL into separate 3-liter bioreactors at an initial culture volume of about 1.2 liters of serum free, chemically defined basal culture medium (n=4). The bioreactors were maintained at 36° C., 6.90 pH, and 350 rpm. Perfusion was initiated on day 1 (or about 24 hours post-inoculation) and used an alternating tangential flow filter system (Repligen, Waltham, MA) with a 30,000 MWCO filter (Cytiva, Westborough, MA). The cultures were perfused with a serum free, chemically defined feed medium. The feed rate and permeate rate were each maintained at 0.13 culture volumes/day (V/d). On day 3, a feed up phase was initiated to increase the culture volume in the bioreactors to a final culture volume of about 1.8 liters, about 100% of the final working volume of the bioreactor. The feed rate was increased to 0.22 V/d and the permeate rate was held constant at 0.13 V/d. On day 4, the feed up phase ended, and the feed rate was decreased to 0.13 V/d, the same as the permeate rate. The viable cell density following the feed up phase was about (20-30)×10⁶ cells/mL. On day 5, the feed rate and permeate rate were increased to 0.22 V/d until harvest. The mAb 4 cultures were harvested on day 15.

In-process samples were taken to monitor culture conditions. Viable cell density (VCD) refers to the number of live cells in a given volume of culture medium. VCD was determined using aCedex HiRes Analyzer (Roche Diagnostics Corporation, Indianapolis, IN). Titer was measured by high performance liquid chromatography (HPLC) via affinity chromatography (Protein A, Waters, Milford, MA).

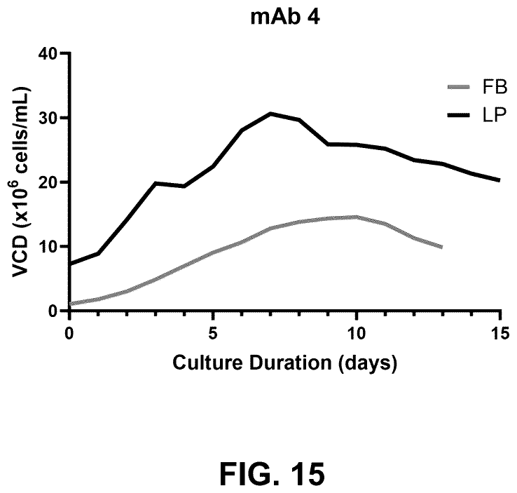

FIG. 15 shows the average viable cell densities (mean±SD) achieved by mAb 4 lean perfusion cultures (solid black line) and fed batch cultures (solid gray line) from initiation to harvest.

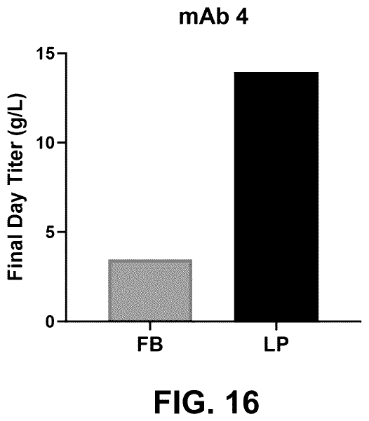

FIG. 16 shows the average final day titer (g/L) for mAb 4 lean perfusion cultures (black column) and fed batch cultures (gray column), with individual run data points shown on each column.

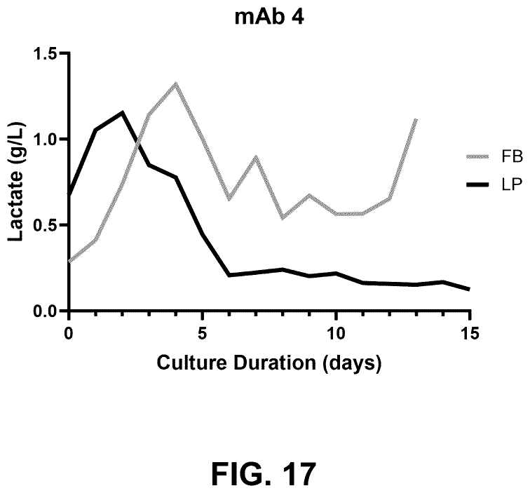

FIG. 17 shows a comparison of average lactate concentration for mAb 4 lean perfusion cultures (solid black line) and fed batch cultures (solid gray line) from initiation to harvest.

The mAb 4 lean perfusion samples showed an approximately 4-fold increase in final day titer compared to their fed batch counterparts, with reduced accumulation of impurities, such as lactate, and increased viable cell densities during the culture.

1. A method for culturing cells to produce a recombinant protein, comprising:
initiating a culture in a bioreactor at a culture volume that is at least 50% of the final bioreactor working volume;
inoculating the culture with cells engineered to express the recombinant protein;
perfusing the culture at one or more perfusion rates of less than or equal to 0.5 culture volumes/day (V/d) until the culture reaches one or more desired target criteria;
increasing the culture volume to a final culture volume; and
once the final culture volume is achieved, perfusing the culture at one or more perfusion rates of less than or equal to 0.5 culture volumes/day (V/d) until the culture is terminated or harvested.

2. (canceled)

3. The method according to claim 1, wherein the culture is initiated at a culture volume that is 60% to 70% of the final bioreactor working volume.

4. The method according to claim 1, wherein the culture is inoculated at a cell density of 5×10⁶ cells/mL to 50×10⁶ cells/mL.

5. (canceled)

6. The method according to claim 1, wherein the bioreactor is operated in batch mode for up to 24 hours following inoculation.

7. The method according to claim 1, wherein the culture is in a growth phase prior to the increase in culture volume to the final culture volume.

8. The method according to claim 7, wherein:
the duration of the growth phase is less than or equal to 60% of the culture duration; and/or
the culture temperature during the growth phase is 35° C. to 37° C.

9. The method according to claim 1, wherein the culture is in a production phase following the increase in culture volume to the final culture volume.

10. The method according to claim 9, wherein the culture temperature during the production phase is 28° C. to 35° C.

11. The method according to claim 1, wherein:
the culture is perfused at one or more perfusion rates of 0.05 V/d to 0.5 V/d until the culture reaches one or more desired target criteria, wherein the feed rate(s) and the contemporaneous permeate rate(s) are the same; and/or
once the final culture volume is achieved, the culture is perfused at one or more perfusion rates of 0.05 V/d to 0.5 V/d until the culture is terminated or harvested, wherein the feed rate(s) and the contemporaneous permeate rate(s) are the same.

12. The method according to claim 1, wherein:
the culture is perfused at one or more perfusion rates of 0.10 V/d to 0.25 V/d until the culture reaches one or more desired target criteria, wherein the feed rate(s) and the contemporaneous permeate rate(s) are the same; and/or
once the final culture volume is achieved, the culture is perfused at one or more perfusion rates of 0.10 V/d to 0.25 V/d until the culture is terminated or harvested, wherein the feed rate(s) and the contemporaneous permeate rate(s) are the same.

13. The method according to claim 1, wherein the one or more desired target criteria are selected from culture volume, final culture volume, bioreactor working volume, final bioreactor working volume, time point, titer, cell density, packed cell volume, product attribute, process, production schedule, plant schedule, and combinations of any of the foregoing.

14. The method according to claim 1, wherein the one or more desired target criteria is a cell density of 100×10⁵ cells/mL to 350×10⁵ cells/mL.

15. The method according to claim 1, wherein the one or more desired target criteria is time post-inoculation, wherein the time post-inoculation is 24 hours to 72 hours.

16. The method according to claim 1, wherein the culture volume is increased to the final culture volume using differential perfusion, wherein the differential perfusion comprises one or more feed rates and one or more permeate rates.

17. The method according to claim 16, wherein at least one of the one or more feed rates is less than or equal to 0.50 V/d and at least one of the one or more permeate rates is less than or equal to 0.20 V/d, wherein each of the one or more feed rates is greater than the contemporaneous permeate rate.

18. The method according to claim 16, wherein at least one of the one or more feed rates is less than or equal to 0.40 V/d and at least one of the one or more permeate rates is less than or equal to 0.15 V/d, wherein each of the one or more feed rates is greater than the contemporaneous permeate rate.

19. (canceled)

20. The method according to claim 16, wherein the differential perfusion continues until the culture volume in the bioreactor is 70% to 100% of the final bioreactor working volume.

21. (canceled)

22. The method according to claim 9, wherein the cell density at the start of the production phase is at least 2 times the inoculation cell density.

23.-28. (canceled)

29. The method according to claim 1, further comprising:
harvesting the recombinant protein;
processing the recombinant protein through one or more unit operations; and
obtaining an isolated, purified recombinant protein.

30.-32. (canceled)

33. The method according to claim 1, wherein the recombinant protein is an antibody.

34. The method according to claim 1, wherein the recombinant protein is an antibody fragment.

35. The method according to claim 1, wherein the cells are CHO cells.
